(19) United States
(12) Patent Application Publication
(10) Pub. No.: US 2007/0298414 A1
(43) Pub. Date: Dec. 27, 2007

(54) Engineering Enzymes Through Genetic Selection

(76) Inventors: Donald F. Doyle, Marietta, GA (US); Lauren J. Schwimmer, San Diego, CA (US); Bahareh Azizi, Atlanta, GA (US)

Correspondence Address: THOMAS, KAYDEN, HORSTEMEYER & RISLEY, LLP, 600 GALLERIA PARKWAY, STE 1500, ATLANTA, GA, 30339, US

(21) Appl. No.: 10/579,683

(22) PCT Filed: Nov. 17, 2004
(86) PCT No.: PCT/US04/38506

§ 371 Date: Jan. 9, 2007

Related U.S. Application Data
(60) Provisional application No. 60/520,754, filed on Nov. 17, 2003.
(60) Provisional application No. 60/520,813, filed on Nov. 17, 2003.
(60) Provisional application No. 60/619,671, filed on Oct. 18, 2004.

(51) Int. Cl. C12Q 1/68; C12N 1/15; C12N 1/16; C12N 15/64; C12N 5/10

(52) U.S. Cl. 435/6; 435/243; 435/254.2; 435/325; 435/471

(57) ABSTRACT

Methods and compositions for selecting transformed cells are provided. An exemplary method combines chemical complementation with genetic selection to identify desirable transformed cells.

### CROSS-REFERENCED TO RELATED APPLICATIONS

This application claims benefit of and priority to U.S. Provisional Patent Application No. 60/520,754 filed on Nov. 17, 2003, U.S. Provisional Patent Application No. 60/520,813, also filed on Nov. 17, 2003, and U.S. Provisional Patent Application No. 60/619,671 filed on Oct. 18, 2004, and where permissible, each of which is incorporated by reference in their entirety.

### STATEMENT REGARDING FEDERALLY SPONSORED RESEARCH OR DEVELOPMENT

Aspects of the work described herein were supported in part by Grant No. DBI-0320786 award by the National Science Foundation. The US government may have certain rights in the disclosed subject matter.

### 1. TECHNICAL FIELD

Aspects of the present disclosure are generally directed to systems and methods for generating ligand-receptor pairs for transcriptional control by small molecules.

### 2. RELATED ART

Directed molecular evolution of enzymes is a developing field in the biotechnology industry and occurs through the single or repeated application of two steps: diversity/library generation followed by screening or selecting for function. The last several years have produced much progress in each of these areas. Techniques of diversity generation in the creation of libraries range from methods with no structure/function prejudice (error-prone PCR; mutator strains) to highly focused randomization based on structural information (site-directed mutagenesis; cassette mutagenesis). DNA recombination (DNA-shufiling, StEP, SCRATCHY, RACHITT, RDA-PCR) requires no structural information but works on the premise that Nature has already solved the problem of creating functional proteins from amino acids. By randomly recombining the genes for related proteins, new combinations of the different solutions are created which may be better than any of the original individual proteins. Structure-based approaches can be combined with other methods to generate greater diversity.

Advances have also been made in screening the generated libraries for proteins with desired properties. In a screen each protein in the library is analyzed for function, which limits library size. In contrast, genetic selection evaluates entire libraries at once, in a highly parallel fashion, because only functional members of the library survive the selective pressure. In selection, nonfunctional members of the library are not individually evaluated. For screens, each variant must be individually assayed and the data evaluated, requiring more time and materials. In vivo genetic selection strategies enable the exhaustive analysis of protein libraries with up to about 10¹⁰ different members. The quoted throughputs are maximal values for industrial, robot driven laboratories. Realistically, experience indicates that an academic, individual investigator laboratory can achieve up to 10⁴ samples/day for screening in yeast and 10⁷ samples/day for genetic selection in yeast. In summary, genetic selection is generally preferable to screening not only because it is higher throughput, but also because it requires less time and materials.

With regard to selection, there are several common conventional selection strategies, such as i) antibiotic resistance, ii) substrate selected growth, where degradation of substrates provides elements essential for growth (such as C, N, P, and S), iii) auxotrophic complementation to restore metabolic function, and iv) phage display, which displays peptides or proteins on a virus surface and segregates them on the basis of binding affinity. Although powerful, these selection strategies are not general enough to apply to engineering enzymes for many interesting reactions. Conventional systems rely on screening techniques rather than selection techniques because selections are more difficult.

The generation of libraries has spawned many companies, in fact, spawned an industry. What has so far failed to be addressed is a general method of evaluating libraries (no matter how they are generated) through genetic selection. Accordingly there is a need for new compositions and methods for engineering polypeptides and rapidly identifying engineered polypeptides having desirable characteristics.

### SUMMARY

Methods and compositions for selecting or screening transformed cells are provided. An exemplary method includes selecting transformed cells by introducing a first polynucleotide into a transformed cell unable to survive on selective media in the absence of a selection agent, wherein the transformed cell expresses a recombinant receptor polypeptide that activates transcription of a second polynucleotide in response to interaction of the recombinant receptor polypeptide with a target substance, culturing the transformed cell on the selective media in the absence of the selection agent; and selecting the transformed cell that survives on the selective media in the absence of the selection agent.

Another aspect provides a method for selecting transformed cells by introducing a first polynucleotide into a transformed cell, wherein the transformed cell expresses a recombinant receptor polypeptide that activates transcription of a second polynucleotide in response to interaction of the recombinant receptor polypeptide with a target substance, culturing the transformed cell on the selective media in the presence of a first selection agent, and selecting the transformed cell that survives on the selective media in the absence of the selection agent, wherein the second polynucleotide encodes an enzyme that converts the first selective agent into a product toxic to the transformed cell.

Still another embodiment provides a cell including a recombinant nuclear receptor that induces transcription of a first polynucleotide in response to interaction with a target substance, and an adapter fusion protein comprising a human coactivator domain operably linked to an activation domain, wherein the adapter fusion protein enhances transcription of the first polynucleotide induced by the recombinant nuclear receptor.

### BRIEF DESCRIPTION OF THE FIGURES

FIG. 1 shows a schematic depicting an exemplary chemical complementation scheme. For selection, yeast strain PJ69-4A has the ADE2 gene under the control of a Gal4 response element (Gal4RE). This strain is transformed with a plasmid expressing ACTR:GAD (manuscript submitted). Plasmids created through homologous recombination in PJ69-4A express a variant GBD:RXR. In media lacking adenine, yeast will grow only in the presence of a ligand that causes the RXR LBD to associate with ACTR and activate transcription of ADE2. For clarity, only one ACTR:GAD is depicted.

FIGS. 2a-o are line graphs showing selection assay (SC-Ade-Trp-Leu+ligand) data for yeast growth in the presence of 9cRA (closed circles) and LG335 (open circles) for 43 hours.

FIGS. 3a-o are line graphs showing screen assay (SC-Trp-Leu+ligand) data for β-galactosidase activity with o-Nitrophenyl β-D-galactopyranoside (ONPG) substrate in the presence of 9cRA (closed circles) and LG335 (open circles). Miller units normalize the change in absorbance at 405 nm for the change optical density at 630 nm, which reflects the number of cells per well.

FIGS. 4a and b are line graphs showing data from mammalian cell culture using a luciferase reporter with wtRXR (solid circle), I268A; I310S; F313A; L436F (solid dot), I268V; A272V; I310M; F313S; L436M (inverted triangle), I268A; I310M; F313A; L436T (gray square), I268V; A272V; I310L; F313M (upright triangle), or I268A; I310A; F313A; L436F (grey circle) in response to (a) 9cRA and LG335 (b). RLU=relative light units.

FIGS. 5a-g are photographs of culture plates showing yeast transformed with both ACTR:GAD and GBD:RXR grow in the presence of various concentrations of 9cRA.

FIGS. 6a-g are photographs of culture plates showing yeast transformed with both SRC-1:GAD and GBD:RXR grow in the presence of various concentrations of 9cRA.

FIGS. 7a-f are photographs of culture plates showing negative selection of yeast transformed with both ACTR:GAD and GBD:RXR in the presence of various concentrations of 9cRA.

FIGS. 8a-t are photographs of culture plates showing growth due to the indicated transformants of variant GBD:RXRs due to various concentrations of 9cRA.

FIGS. 9a-e are schematics of exemplary embodiments for the selection of desired transformants.

FIG. 10 is a schematic of an exemplary embodiment for the selection of selective receptor modulators in transformants incorporating a human nuclear receptor coactivator fused to a repression domain.

FIG. 11 is a schematic of an exemplary embodiment for the selection of receptor antagonists.

FIG. 12 is a schematic of an exemplary embodiment for chemical complementation selection of transformants to obtain isotype or isoform selective receptor agonists.

FIG. 13 is a schematic of an exemplary embodiment for chemical complementation selection of transformants incorporating a nuclear receptor coactivator fused to an activation domain for the selection of receptor agonists.

FIG. 14 is a Ligplot depiction of hydrophobic interactions between the RXR LBD and 9cRA.

FIGS. 15a-b show the structure of exemplary ligands used in chemical complementation of one embodiment.

FIGS. 16a-b show schematics of exemplary methods for the construction of pGBDRXR:3stop (a) or an insert cassette library (b).

FIGS. 17a-b are diagrams of exemplary constructs according to one embodiment of the present disclosure.

### DETAILED DESCRIPTION

Methods and compositions for engineering proteins are provided, in particular, methods for engineering proteins that interact with a target compound. Embodiments of the disclosure combine chemical complementation with genetic selection to engineer proteins, polypeptides, enzymes, antibodies, adhesins, integrins, and the like. Typically, any protein or polypeptide that interacts with a small molecule can be engineered or modified using the disclosed methods and systems. Exemplary proteins include, but are not limited to enzymes, antibodies, cell surface receptors, polypeptides involved in signal transduction pathways, intracellular polypeptides, secreted polypeptides, and transmembrane polypeptides. In some embodiments, the polypeptides interact with a small molecule that is produced naturally. Representative naturally produced small molecules include but are not limited to, neurotransmitters, cAMP, cGMP, steroids, purines, pyrimidines, heterocyclic compounds, ATP, DAG, IP3, inositol, calcium ions, magnesium ions, vitamins, minerals, and combinations thereof. Some embodiments provide methods and systems for engineering proteins that distinguish between optical isomers of a target compound.

Other embodiments provide a more efficient mammalian model system in yeast for evaluating protein/ligand interactions, and can be utilized in an array of applications including but not limited to drug discovery. Nuclear receptors are implicated in diseases such as diabetes and various cancers. Agonists and antagonists for these nuclear receptors serve as drugs. With chemical complementation, libraries of compounds can be screened as potential agonists, as described herein. In some embodiments, antagonists can be identified with negative chemical complementation. Chemical complementation can also be extended to identify isotype-selective agonists and antagonists and used for the discovery of selective receptor modulators (e.g., SERMs).

In addition to drug discovery, the increase in sensitivity of disclosed systems and methods also provides a method for engineering receptors to recognize small molecules. For example, libraries of engineered receptors can be transformed into yeast and plated onto media containing the target ligand. These engineered receptors can be used for controlling transcription in mammalian cells, and potentially applied towards gene therapy. Furthermore, some embodiments of the disclosed system can give insight into the general mechanism for understanding the fundamentals of protein structure and function.

In summary, we have demonstrated that the addition of an adapter protein consisting of a human coactivator fused to a yeast transcriptional activator increases the sensitivity of chemical complementation with RXR 1000-fold, enhancing the system so that it is indistinguishable from activation by Gal4. Negative chemical complementation was performed in a different yeast strain, showing the versatility of the system, useful for performing chemical complementation with various selectable markers. This system may be extended to the ˜75 human nuclear receptor proteins, plus nuclear receptors from other organisms, and the coactivators and corepressors with which they interact.

Embodiments of the present disclosure comprise chemical complementation systems focusing on one small molecule target ligand and utilize the power of genetic selection to reveal proteins within the library that bind and activate transcription in response to that small molecule. Functional receptors from a large pool of non-functional variants can be isolated, even from a non-optimized library.

Chemical complementation is a method which links survival of yeast to the presence of a small molecule. This process allows high-throughput testing of large libraries. Hundreds of thousands to billions of variants can be assayed in one experiment without the spatial resolution necessary for traditional screening methods (e.g., no need for one colony per well). Yeast can be spread on solid media and, through the power of genetic selection, cells expressing active variants will grow into colonies. Survivors can then be spatially resolved (e.g. transferred to a microplate, one colony per well) for further characterization, decreasing the time and effort required to find new ligand-receptor pairs.

In one embodiment, among others, chemical complementation identifies nuclear receptors with a variety of responses to a specific ligand. Nuclear receptors that activate transcription in response to targeted molecules and not to endogenous compounds have several additional potential applications. The ability to switch a gene on and off in response to any desired compound can be used to build complex metabolic pathways, gene networks, and to create conditional knockouts and phenotypes in cell lines and animals. This ability can also be useful in gene therapy and in agriculture to control expression of therapeutic, pesticidal, or other genes. A variety of responses would be useful in engineering biosensor arrays: an array of receptors with differing activation profiles for a specific ligand could provide concentration measurements and increased accuracy of detection.

The ability to engineer proteins that activate transcription in response to any desired compound with a variety of activation profiles will provide a general method of identifying enzymes. Receptors that bind the product of a desired enzymatic reaction can be used to select or screen for enzymes that perform this reaction. The enzymes may be natural or engineered. The stringency of the assay can be adjusted by using ligand-receptor pairs with lower or higher EC₅₀. The lack of a general system for genetic selection is currently the limiting step for directed evolution of enzymes.

The human retinoid X receptor (RXR) is a ligand-activated transcription factor of the nuclear receptor superfamily. RXR plays an important role in morphogenesis and differentiation and serves as a dimerization partner for other nuclear receptors. Like most nuclear receptors, RXR has two structural domains: the DNA binding domain (DBD) and the ligand binding domain (LBD), which are connected by a flexible hinge region. The DBD contains two zinc modules, which bind a sequence of six bases. The LBD binds and activates transcription in response to multiple ligands including phytanic acid, docasahexaenoic acid and 9-cis retinoic acid (9cRA). RXR is a modular protein; the DBD and LBD can function independently. Therefore, the LBD can be fused to other DBDs and retain function. A conformational change is induced in the LBD upon ligand binding, which initiates recruitment of coactivators and the basal transcription machinery resulting in transcription of the target gene.

Nuclear receptors have evolved to bind, and activate transcription in response to, a variety of small molecule ligands. The known ligands for nuclear receptors are chemically diverse, including steroid and thyroid hormones, vitamin D, prostaglandins, fatty acids, leukotrienes, retinoids, antibiotics, and other xenobiotics. Evolutionarily closely related receptors (e.g., thyroid hormone receptor and retinoic acid receptor) bind different ligands, whereas some members of distant subfamilies (e.g., RXR and retinoic acid receptor) bind the same ligand. This diversity of ligand-receptor interactions demonstrates the versatility of the fold for ligand binding and suggests that it should be possible to engineer LBDs with a large range of novel specificities.

The crystal structure of RXR bound to 9cRA elucidates important hydrophobic and polar interactions in the LBD binding pocket. In one embodiment, a subset of 20 hydrophobic and polar amino acids within 4.4 Å of the bound 9cRA are varied to make a library. These residues in RXR are good candidates for creating variants that bind different ligands through site directed mutagenesis, because side chain atoms, not main chain atoms, contribute the majority of the ligand contacts. A library of RXR LBDs with all 20 amino acids at each of the 20 positions in the ligand-binding pocket screened against multiple compounds could potentially produce many new ligand-receptor pairs. However, the number of possible combinations (20²⁰≈10²⁶) renders saturation mutagenesis impractical for constructing a complete library.

Codon randomization creates protein libraries with mutations at specific sites. In one embodiment, a modified version of the Sauer codon randomization method to create a library of binding pocket variants of RXR is provided. This library allowed exploration of a vast quantity of sequence space in a minimal amount of time.

Chemical complementation allows testing for the activation of protein variants by specific ligands using genetic selection. In one embodiment LG335 was used, a synthetic retinoid-like compound, as a model for discovery of ligand-receptor pairs from large libraries using chemical complementation. LG335 was previously shown to selectively activate an RXR variant and not activate wild-type RXR. Combining chemical complementation with a large library of protein variants decreases the time, effort, and resources necessary to find new ligand-receptor pairs.

Enzyme Engineering

One embodiment provides methods and compositions for engineering a polypeptide, for example an enzyme, to produce or interact with a desired molecule. Generally, a desired molecule of interest (or the reaction product) is chosen, and a target nuclear receptor is also chosen. After the target molecule and the target nuclear receptor are selected, modifications to the target nuclear receptor can be designed. For example, the X-ray structure of the target nuclear receptor can be loaded into a modeling program, including, but not limited to Insight® or Flexx®, along with the structure of the desired target molecule. Specific in silico interactions of the target receptor with the target molecule/ligand can be analyzed and those amino acids that may contribute the ligand binding can be noted for modification. Generally, a nuclear receptor is selected that has at least a detectable amount of interaction with the target molecule or ligand or a binding pocket of a similar size and shape. The interaction can then be modulated as desired by creating a library of modified receptors.

To create the library, site-specific codon randomization can be used. It will be appreciated that any process for generating a library of modified receptors can be used. Site-specific codon randomization involves modifying the amino acids identified through modeling as having or believed to have direct or indirect interactions with the ligand. When producing or designing the oligonucleotide, in place of those amino acids, there will be a degenerate code based on the combination of nucleotides that are desired. For example, if the modification can be a change from alanine to a cysteine, leucine, phenylalanine, isoleucine, threonine, serine, valine and methionine. The nucleotide sequence for the alanine is GCC and to possibly incorporate all of the desired amino acids mentioned above, the following changes in each position must be made:

| G | C | C |
|---|---|---|
| 1 | 2 | 3 |
| T | T | |
| A | | |
| G | | G |
| | C | C |

The oligonucleotide can be designed to have either a T, A, or G in the first position, a T or C in the second position, and a G or C in the third position. For example, if a TTG (one of the combinations above) is in place of the GCC, that would incorporate a leucine instead of the alanine. Therefore, when the oligos are ordered, you would order them such that you get the possibility of a T, A, or G in the first position, a T or C in the second position, and a G or C in the third position. The oligonucleotides may be designed to include insertions or deletions. The oligonucleotides have ends that are homologous to the vector in which the gene will be introduced to.

In one embodiment, to create a receptor library, the vector into which the gene will be incorporated will be cut with restriction enzymes, deleting a fragment of the wild-type gene. Oligonucleotides will be designed with homologous ends to the vector as mentioned above, but these oligonucleotides will also be designed such that they overlap each other. The overlapping ends will hybridize to each other, and using for example the enzyme Klenow, the ends are filed in. Then using the polymerase chain reaction (PCR) the full gene or a fragment thereof will be amplified. After both of these products are made, these genes will be introduced into chemical complementation. The vector and gene will be introduced into yeast using transformation protocols, for example protocols introduced by Gietz and co-workers. During transformation, the vector and gene or gene fragment will homologously recombine, and the various receptor mutants will be expressed.

To select for variants that bind the desired small molecule, chemical complementation is be used. Chemical complementation is a general method of linking any small molecule to genetic selection. Chemical complementation is a new derivative of the yeast two-hybrid system, a three-component system that in one embodiment comprises a human nuclear receptor protein, its coactivator protein, and a small molecule ligand, where the nuclear receptor and coactivator associate and activate transcription only in the presence of the ligand. An exemplary yeast strain contains a Gal4 response element fused to the ADE2 gene. If adenine is not provided in the medium, the yeast will not be able to survive unless they are able to make their own, and to do that, expression of ADE2 needs to be activated. The following exemplary plasmids can be utilized: 1^{st} plasmid encodes a fusion protein of the Gal4 DNA binding domain (Gal4 DBD) fused to the variant receptor ligand-binding domain (LBD); the other fusion protein comprises a human coactivator protein fused to the Gal4 activation

[image]

domain. In the presence of ligand, the ligand will bind to the variant receptor ligand-binding domain and the Gal4 DNA binding domain will bind to the Gal4 response element. This will cause the protein to undergo a conformational change, and will recruit the coactivator fused to the Gal4 activation domain. This, in turn, will result in RNA polymerase being recruited and activation of transcription of the downstream gene.

The transformed yeast from above will be plated onto plates containing the desired small molecule. Through chemical complementation, the variant receptor that is able to bind the desired molecule and activate the ADE2 gene allowing that yeast colony to grow. The plasmid from that colony will be rescued and sequenced and an engineered receptor will be identified and will be carried on to the next step. It will be appreciated that there may be many variant receptors that allow the yeast to grow without binding the targeted ligand. For example, they may be constitutively active or bind an endogenous small molecule. These receptors may be identified through screening without the targeted ligand. Alternatively, they may be removed from the library by negative genetic selection on media without the targeted ligand, either before or after chemical complementation. Once an engineered receptor has been created, this gene can be integrated into the yeast genome, for example via homologous recombination. This will create a new strain that will be used in the following process.

Once the receptor that can bind the small molecule has been identified, individual enzymes or a library of enzymes can be evaluated to generate the product of interest. Libraries of naturally occurring enzymes, for example expression cDNA libraries, may be evaluated. Also, libraries of enzymes can be created using a number of mutagenic protocols, such as DNA shuffling, RACHITT, Error-Prone PCR, to name a few. For example, an enzyme that is suspected of interacting with the target molecule can be selected and mutagenized with conventional techniques. Alternatively, yeast or microorganisms can be randomly mutated.

In one embodiment, chemical

[image]

complementation is used to identify the engineered enzyme. In this embodiment the library of engineered enzymes will be introduced into the yeast strain transformed with the modified nuclear receptor described above. This yeast strain has a variant receptor integrated into its genome, and the variant receptor is able to bind the product molecule. Once the engineered enzymes have been transformed into the yeast strain, the yeast will be spread onto selective plates (for example plates lacking adenine) containing the reactants involved in the enzymatic reaction that can be used to synthesize the missing product. The yeast will be able to take the reactants and if the yeast express an engineered enzyme that can convert the reactants to the reaction product, then the yeast will survive. The yeast will survive because the reaction product will be able to bind to the variant receptor, and activate transcription of the ADE2 gene or other selection gene. The DNA from the yeast colony that grew will be rescued and sequenced.

Target compounds that serve as ligands can be selected from any variety of natural or synthetic compounds. In one embodiment, natural products with agricultural or medicinal applications can be selected as target compounds. The search for natural products as potential agrochemical agents has increased due to the demand for crop protection chemicals. In 1990, the world market value of pesticides totaled nearly $23 billion. Synthetic chemical pesticides are used to protect crops but several developments have triggered the search for alternative compounds. First, resistance has developed against synthetic chemical pesticides. Second, concern has arisen regarding potential human health risks. Third, there is a growing awareness of environmental damage, such as contamination of soil, water, and air. New environmentally friendly methods are being pursued to rectify these problems. In one embodiment of the present disclosure, the disclosed methods can be used to identify new prototype pesticides in natural products produced by microorganisms, for example, which

[image]

are perceived as more environmentally friendly and acceptable. The natural products would be applied as the synthetic chemical pesticides have been or the biosynthetic genes would be expressed in transgenic plants. This strategy has been widely applied using the Bacillus thuringiensis toxin. In another embodiment, genes for toxins are delivered to target pest species using insect-specific viruses that leave beneficial insects unharmed. These “greener” technologies require not only identification of active natural products but also the genes for their biosynthesis. With these applications in mind, and because of their availability, three compounds have been chosen as target ligands. Barbamide and jaspamide are relevant to the agricultural industry. Resveratrol has antiviral, antimicrobial, and anticancer effects.

Barbamide is a natural product from the marine cyanobacterium, Lyngbya majuscula. From 295 g of algae, 258 mg of pure barbamide can be isolated. This chlorinated lipopeptide has potent mollucuscidal activity. The gene cluster for barbamide biosynthesis from L. majuscula has been cloned and analyzed. An ˜26 kb region of DNA from this organism specifies the biosynthesis of barbamide. The gene cluster revealed 12 open reading frames and it is believed that barbamide is synthesized from acetate, L-phenylalanine, L-cysteine, and L-leucine. Polyketide synthase and non-ribosomal peptide synthetase modules accomplish biosynthesis. A trichloroleucine intermediate is involved, but an unresolved issue is its transfer between modules. The total synthesis of barbamide has been reported.

Jaspamide was isolated from various marine sponges and exhibits insecticidal (against Heliothis virescens) and fungicidal activity (against Candida albicans). It is completely inactive against a series of Gram negative and Gram-positive bacteria. From 700 g of sponge tissue, 80 mg of pure jaspamide was isolated. The biosynthetic pathway has not been elucidated, but its structure suggests polyketide synthase and non-ribosomal peptide synthetase modules. Since it is a fungicide, a bacterial chemical complementation system for engineering nuclear receptors and discovering the genes involved in the biosynthesis of this compound would be used.

Resveratrol is a stilbene phytoalexin that is produced in at least 72 plant species. Phytoalexins are low molecular weight antimicrobial metabolites that are produced by plants for protection against a wide range of pathogens. Some nuclear receptors are known to bind resveratrol, making the DNA shuffling approach to engineer a receptor highly relevant. This compound is commercially available on the gram scale. [symbol] [image] enzymes such as formate dehydrogenase (FDH).

The starting enzyme is typically examined for, albeit small, levels of activity against a substrate, for example the ketone substrate in a high ammonia environment, either i) in water/liquid ammonia-mixtures, or ii) in saturating concentrations of ammonium formate or ammonium carbonate. A sensitive assay can be employed to check for NADH consumption such as formation of formazan (λₘₐₓ=450 nm). In this embodiment, an (S)-amino acid dehydrogenase, either PheDH from Rhodococcus rhodocrous or LeuDH from Bacillus stearothermophilus, an (R)-AmDH can be developed through change of substrate specificity. Diversity is generated within the respective gene through both random mutagenesis and recombination. Selection via binding of the product to a nuclear receptor with subsequent transcriptional control is chosen as the strategy to assay for successful variants.

Nuclear receptors PXR, BXR, and RAR can be used for engineering (R)-amine activated transcription with the disclosed methods and compositions. For example, these nuclear receptors can be engineered to activate the transcription of the essential metabolic gene ADE2 in response to the (R)-amines in the modified Saccharomyces cerevisiae strain PJ69. PXR is chosen because of its broad substrate specificity. BXR is chosen because it is already known to activate transcription in response to amines. Random and structure-based approaches of creating libraries to engineer the nuclear receptors for (R)-amine activated growth through genetic selection can be used. Receptors for multiple (R)-amines will be engineered in parallel by selecting each library on multiple selective plates with the appropriate (R)-amine. Optionally, negative selection to genetically select libraries against enzymes that make an S-enantiomer product then select for the production of the R-enantiomer (or vice-versa) can be used. A nuclear receptor library for the (R)-amine ligand can be synthesized. Additionally, the (R)-amine ligand can be synthesized in vivo by an expressed AmDH from the ketone precursor supplemented within the growth medium. A mutant PheDH library can then be screened for in vivo synthesis of (R)-amines. In this overall scheme, the power of genetic selection is used to detect biocatalytic synthesis of amines. Utilizing genetic selection means that each member of the library does not need to be screened, only functional AmDH appear because they allow the microbe to grow and form a colony. Furthermore, catalysis is directly selected, as opposed to some related but indirect property (like transition state binding). Genetic selection coupled with the broad ligand specificity of nuclear receptors creates a process to rapidly improve biocatalysts for more efficient synthesis of enantiomerically pure compounds.

Selected transformants can be optimized through successive rounds of directed evolution. Further mutant libraries of PheDH/LeuDH enzymes can be screened for in vivo synthesis of (R)-amine. Mutant AmDH enzymes can be expressed and further studied for shifts in substrate specificity and changes in kinetic reaction rates.

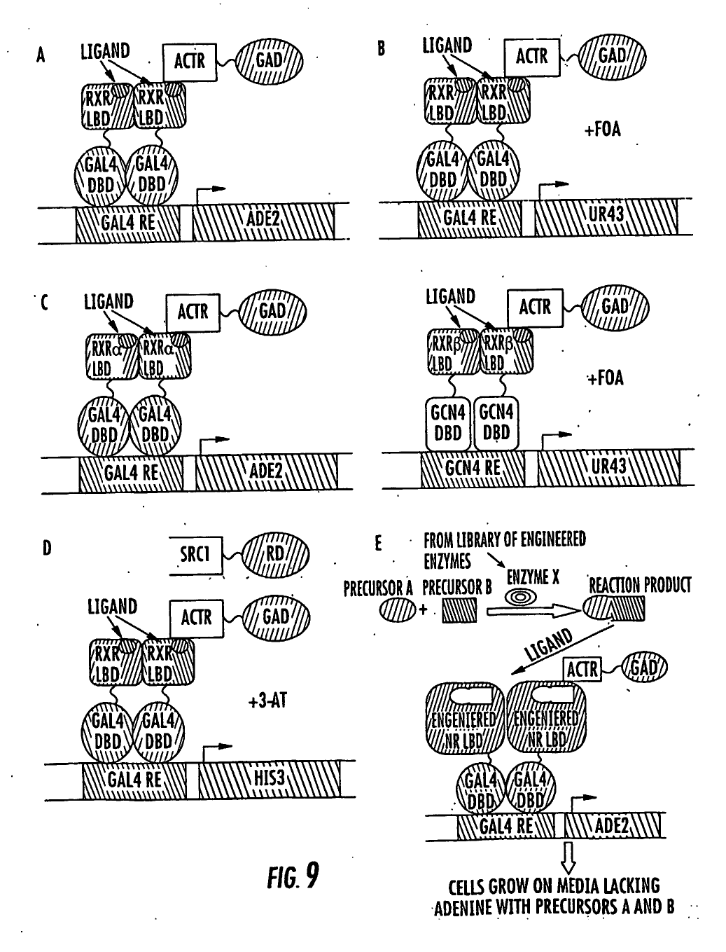

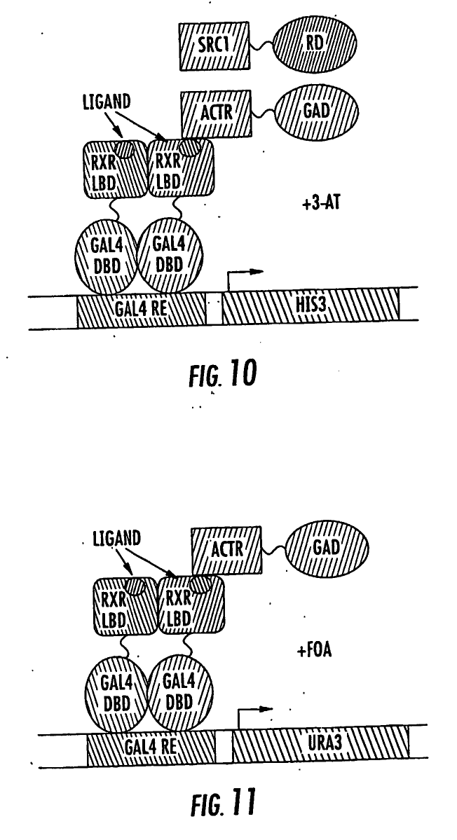

FIG. 10 depicts another embodiment for the identification of selective receptor modulators (analogous to selective estrogen modulators). In this embodiment, the human nuclear receptor coactivator ACTR is fused to the Gal4 activation domain (ACTR:GAD). Additionally, the human nuclear receptor coactivator SRC1 is fused to a yeast repression domain (SRC1:RD). In the presence of an agonist, these coactivator fusion proteins compete for expression of the HIS3 gene. The HIS3 gene encodes imidazoleglycerolphosphate dehydratase. In the presence of an agonist that recruits both coactivators equally, the yeast probably will produce enough histidine to survive. Adding the inhibitor 3-AT to the plates raises the threshold of enzyme that must be produced to permit growth. Compounds that selectively favor the RXR-ACTR interaction over the RXR-SRC-1 interaction will allow yeast to grow.

FIG. 11 is a diagram of another embodiment incorporating negative chemical selection. Human nuclear receptor coactivator, ACTR is fused to the Gal4 activation domain (ACTR:GAD). The Gal4 DBD is fused to the nuclear receptor LBS (GBD:RXR). The Gal4 DBD binds to the Gal4 response element, regulating transcription to the URA3 gene. The URA3 gene codes for orotidine-5′-phosphate decarboxylase, an enzyme in the uracil biosynthetic pathway. This gene can be used for both positive and negative selection. For positive selection, yeast expressing this gene will survive in the absence of uracil in the media. For negative selection, 5-fluoroorotic acid (FOA) is added to the media. Expression of orotidine-5′-phosphate decarboxylase coverts FOA to the toxin 5′-fluorouracil, which kills the yeast. Libraries of small molecules can be screened in a high-throughput assay in wells containing an agonist and FOA. Antagonists will allow yeast to grow.

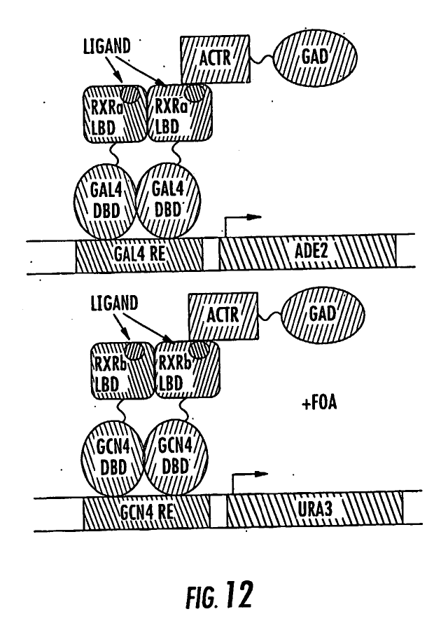

FIG. 12 is a diagram illustrating still another embodiment comprising isotype specific nuclear receptor agonists are. Each isotype can be fused to a different DBD controlling expression of different genes. The isotype for which an agonist is sought is fused to the Gal4 DBD to control expression of ADE2 (for positive chemical complementation). The isotype against which selectivity is desired, is fused to the GCN4 DBD to control expression of the URA3 gene (for negative chemical complementation). Libraries of small molecules are screened in individual wells of a 384-well plate. Compounds that do no activate the receptor will no allow the yeast to grow. Compounds that agonize both isotypes will kill the yeast. Only compounds that agonize RXRα, and either do not bind or antagonize RXRβ will allow yeast to grow.

FIG. 13 shows another embodiment in which a human nuclear receptor coactivator, ACTR, is fused to the Gal4 activation domain (ACTR:GAD). The Gal4 DBD is fused to the nuclear receptor LBD (GBD:RXR). The Gal4 DBD binds to the Gal4 response element, regulating transcription of the ADE2 gene. Upon binding of the ligand, the LBD of the nuclear receptor undergoes a conformational change, which recruits the ACTR:GAD fusion protein. This brings the Gal4 AD and Gal4 DBD into close proximity activating transcription of the ADE2 gene. For clarity only one ACTR:GAD protein is shown binding one GBD:RXR. Libraries of small molecules are screened in individual wells of a 384-well plate. Agonists will allow yeast to grow.

Materials and Methods

Ligands. 9-cis retinoic acid (MW=304.44 g/mol) was purchased from ICN Biomedicals.

LG335 Synthesis

### 3-(1-Carbonyl)propyl-5,5,8,8-tetramethyl-5,6,7,8-tetrahydronapthylene

2,5-dimethyl-2,5,hexanediol (5.0 g, 34 mmol) was dissolved in anhydrous benzene (150 mL). AlCl₃ (5.0 g, 38 mmol) was added slowly while the mixture was stirred in an ice bath, followed by stirring at room temperature for 1 hour. Another portion of AlCl₃ (5.0 g, 38 mmol) was then added and the reaction was heated to 50° C. and stirred overnight. The brown solution was poured over iced 0.4 M HCl (50 mL) and extracted with ether (3×50 mL). The organic layer was then sequentially washed with water, saturated aqueous NaHCO₃, and brine (80 mL each) and dried (MgSO₄). The solvent was removed in vacuo to afford 6.2 g of a yellow liquid (2).

The crude product was then mixed with propionyl chloride (3.2 mL, 37 mmol) and the resulting solution added dropwise to a mixture of AlCl₃ (5.0 g, 38 mmol) in dichloroethane (20 mL) while maintaining the temperature between 20 and 25° C. The mixture was stirred for 2 hours at room temperature, at which point it was quenched by pouring carefully over ice. The reaction mixture was then extracted methylene chloride (3×10 mL). The organics layers were then combined, washed with water and saturated aqueous NaHCO₃ the volatiles removed by rotary evaporation. The product was purified by silica gel column chromatography eluting with hexanes:chloroform (4:1, then 1:1) to yield 6.9 g (28 mmol, 73%) of product as a yellow oil (3, 4).

### 3-Propyl-5,5,8,8-tetramethyl-5,6,7,8-tetrahydronapthylene

3-(1-Carbonyl)propyl-5,5,8,8-tetramethyl-5,6,7,8-tetrahydronapthylene (1.0 g, 4.1 mmol) in MeOH (10 mL), H₂O (1 mL), and conc. HCl (3 drops) was treated with 10% Pd/C (144 mg) and subjected to catalytic hydrogenation conditions at 60 psi while heating gently overnight. When the reaction was considered complete (Rf=0.76, 5% EtOAc in hexanes) it was filtered through a celite pad and rinsed with MeOH (10 mL) and hexane (50 mL). Water (1 mL) was then added to the filtrate and the organic phase separated and washed with brine (2×20 mL). The aqueous layer was washed with hexanes (2×20 mL). The organic layers were dried (Na₂SO₄), filtered and the volatiles removed by rotary evaporation to produce 510 mg (2.2 mmol, 54%) of a colorless oil (5).

### 4-[(3-Propyl-5,5,8,8-tetramethyl-5,6,7,8-tetrahydro-2-naphtyl) carbonyl]benzoic Acid (LG335)

3-Propyl-5,5,8,8-tetramethyl-5,6,7,8-tetrahydronapthylene (2.2 g, 9.5 mmol) and chloromethyl terephthalate (2.0 g, 10 mmol) were dissolved in dichloroethane (20 mL) and FeCl₃ (80 mg, 490 μmol) was added. The reaction mixture was stirred at 75° C. for 24 hours. The reaction was then cooled and MeOH (20 mL) added. The resulting slurry stirred for 7 hours at room temperature, filtered and rinsed with cold MeOH (20 mL) to result in 2.1 g (5.5 mmol, 58%) of white crystals (6).

The crystals (107 mg, 280 μmol) were stirred in MeOH (2 mL), to which 5N KOH (0.5 mL) was added. This mixture was refluxed for 30 minutes, cooled to room temperature and acidified with 20% aqueous HCl (0.5 mL). The MeOH was evaporated and the residue was extracted with EtOAc (2×5 mL). The organic layers were combined and dried (MgSO₄) and filtered. The filtrate was treated with hexane (10 mL) and reduced in volume to 2 mL. After standing overnight the resulting crystals were collected to provide 39 mg (103 μmol, 37%) as a white powder (1). mp 250-252° C.; H¹ NMR (CDCl₃) δ 0.88 (t, 3H, —CH₂CH₂CH₃), 1.20 (s, 6H, CH₃),1.32 (s, 6H, CH₃), 1.55 (dt, 2H, —CH₂CH₂CH₃), 1.69 (s, 4H, CH₂), 2.65 (t, 2H, —CH₂CH₂CH₃), 7.20 (s, 1H, Ar—CH) 7.23 (s, 1H, Ar—CH), 7.89 (d, 2H, Ar—CH), 8.18 (d, 2H, Ar—CH); MS (EI POS) m/z mass for C₂₅H₃O₃: Calc. 378.2189, Found 378.2195; Anal. for C₂₅H₃O₃: Calc. C, 79.33; H, 7.99, Found C, 79.10; H, 7.96.

Expression Plasmids. pGAD10BAACTR, pGBT9Gal4, pGBDRXRα, pCMX-hRXR, and pCMX-βGAL have been described. pCMX-hRXR mutants were cloned from pGBDRXR vectors using Sall and Pstl restriction enzymes and ligated into similarly cut pCMX-hRXR vectors. pLuc_CRBPII_MCS was constructed as below. All plasmids have been confirmed through sequencing.

pGBDRXRα was cut with Smal and Ncol, filled in, and blunt-end ligated to eliminate 153 amino acids of the RXR DBD. A HindIII site in the tryptophan selectable marker was silently deleted and the sole remaining HindIII site was cut, filled in, and blunt-end ligated to remove the restriction site. Unique HindIII and Sacl sites were inserted into the RXR LBD gene and Mfel and EcoRI sites were removed from the plasmid using QuikChange Site-Directed Mutagenesis (Stratagene, La Jolla, Calif.) to create pGBDRXRαL-SH-ME.

pLuc_CRBPII_MCS was made by site-directed mutagenesis from pLucMCS (Stratagene, USA). Site-directed primers were designed to incorporate a CRBPII response element in the multiple cloning site (MCS), controlling transcription of the firefly luciferase gene.

Plasmids expressing the fusion protein of the Gal4 activation domain with the coactivators are based on the commercial plasmid pGAD10 (Clontech, USA). The pGAD10 vector contains the Gal4 activation domain (residues 491-829) fused to a multiple cloning site (MCS) and uses a leucine marker. Additional restriction enzyme sites were added to the MCS of the plasmid via site directed mutagenesis Primers were designed to add the following restriction enzymes: NdeI, EagI, ECIXI, NotI, XmaIII, XmaI, and SmaI, forming a new plasmid known as pGAD10BA. (FIG. 17) This plasmid was sequenced and used for specific interaction studies mentioned in the results.

pCMX-ACTR, the expression plasmid for the human nuclear receptor coactivator ACTR, was a kind gift from Dr. Ron Evans (Salk Institute for Biological Studies, La Jolla, Calif.). pCR3.1 hSRC-1, the expression plasmid for the human nuclear receptor coactivator SRC-1, was a kind gift from Dr, Bert O'Malley (Baylor College of Medicine, Houston, Tex.). Both ACTR (residues 1-1413) and SRC-1 (residues 54-1442) genes were amplified via PCR with primers that contained BgIII and NotI sites. The PCR products were digested with the two restriction enzymes and cleaned using the Zymo “DNA Clean and Concentrator Kit” (Zymo Research, Orange, Calif.) spin columns, pGADIOBA was digested with BgIII and NotI and ligated with both the ACTR and SRC-1 products. Ligations were transformed into Z-competent (Zymo Research, Orange, Calif.) XL 1-Blue cells (Stratagene, La Jolla, Calif.). Transformants were rescued and sequenced. The final plasmids are called pGAD10BAACTR and pGAD10BASRC1.

Plasmid Construction. The zero background plasmid, pGBDRXR:3Stop, was constructed using QuikChange Site-Directed Mutagenesis with pGBDRXRαL-SH-ME as the template and the 3Stop insert cassette (described below) as primers.

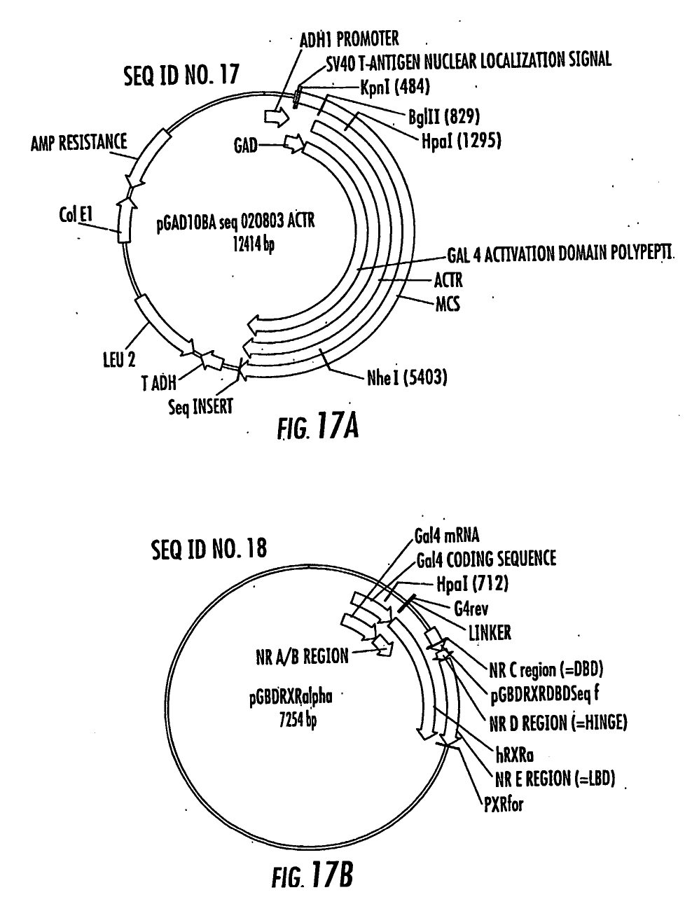

The 3Stop insert cassette was synthesized using PCR from eight oligonucleotides (FIG. 16). All PCRs were done using 2.5 U Pfu Polymerase (Stratagene, LaJolla, Calif.), 1× Pfu buffer, 0.8 mM dNTPs, 50 ng of pGBDRXRαL-5H-ME as a template, 125 ng of primers and sterile water to make 50 μL. First, four small cassettes were synthesized in reactions containing the following primers: Cassette 1, F (5′-CGGAATTTCC CATGGGC-3′) (SEQ ID NO. 1), BPf (5′-CTCGCCGAAC GACCCGGTCA CCGCATGCCA CTAGTGG-3′) (SEQ ID NO. 2), and BPr (5′-CCGCTTGGCC CACTCCACTA GTGGCATGCG GTGACC-3′) (SEQ ID NO. 3); Cassette 2, BPf, BPr, SEf (5′-CGGGCAGGCT GGAATGAGCT CCTCGACGGA ATTCTCC-3′) (SEQ ID NO. 4), and SEr (5′-CAGCCCGGTG GCCAGGAGAA TTCCGTCGAG GAGCTC-3′) (SEQ ID NO. 5); Cassette 3, SEf, SEr, AMf (5′-CTCTGCGCTC CATCGGGCTT AAGTGCCCAC CAATTGACAC-3′) (SEQ ID NO. 6), and AMr (5′-CTCCAGCATC TCCATAAGGA AGGTGTCAAT TGGTGGGCAC TTAAGC-3′) (SEQ ID NO. 7); Cassette 4, AMf, AMr, and R (5′-CAAAGGATGG GCCGCAG-3′) (SEQ ID NO. 8). The cassettes were cleaned with either the DNA Clean and Concentrator-5 (Zymo Research, Orange, Calif.) or the Zymoclean Gel DNA Recovery Kit (Zymo Research, Orange, Calif.) depending on product purity. The four cassettes were used to make the final 3Stop insert cassette in a PCR that contained each cassette, primers F and R, dNTPs, Pfu Polymerase, and sterile water to a final volume of 50 μL. The 3Stop cassette was cleaned using the Zymoclean Gel DNA Recovery Kit.

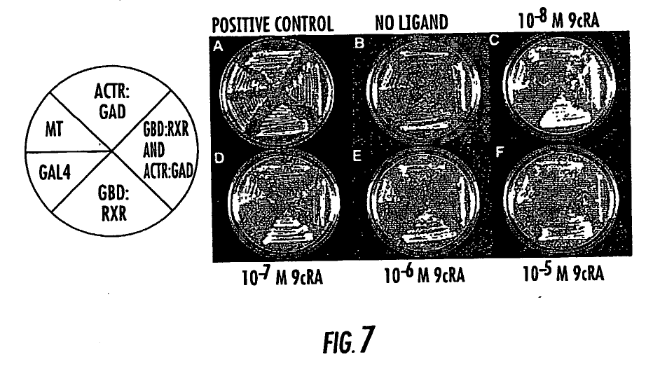

Insert Cassette Library Construction. The library of insert cassettes with randomized codons was constructed in a similar manner as above. The four cassettes (FBP, BPSE, SEAM and AMR) were made in the following ways (Supporting Information FIG. 7b).

For the FBP cassette, oligos BP1 (5′-GGCAAACATG GGGCTGAACC CCAGCTCGCC GAACGACCCG GTCACC-3′) (SEQ ID NO. 9), BP2 (5′-GCCCACTCCA CTAGTGTGAA AAGCTGTTTG TC (A, C, or T)(A or G)(C or G)(A, C, or T)(A or G)(C or G)TT GGCA(A, C, or T)(A or G)(C or G)GTT GGTGACCGGG TCGTTCG-3′) (SEQ ID NO. 10), BP3 (5′-CTTTTCACAC TAGTGGAGTG GGCCAAGCGG ATCCCACACT TCTCAGAG-3′) (SEQ ID NO. 11), and BP4 (5′-GGGGCAGCTC TGAGAAGTGT GGGATCCG-3′) (SEQ ID NO. 12) were mixed with TE containing 100 mM NaCl to bring the total volume to 50 μL. The mixture was heated to 95° C. for 1 minute, then slowly cooled to 10° C. The annealed mixture was combined with EcoPol Buffer, dNTPs, ATP, Klenow (NEB, Beverly, Mass.), T4 DNA ligase (NEB, Beverly, Mass.) and sterile water to 200 μL, and kept at 25° C. for 45 min before heat inactivation at 75° C. for 20 minutes. The product was cleaned with DNA Clean and Concentrator-5 to make the BP cassette. Next, BP cassette was combined with Pfu Buffer, pGBDRXR:3Stop, oligo F, dNTPs, Pfu polymerase, and sterile water to make 50 μL for a PCR. The final FBP product (300 bp) was purified using the Zymoclean Gel DNA Recovery Kit.

BPSE was made in two consecutive PCRs. First, SE1 (5′-GCAGGCTGGA ATGAGCTCCT C(A, G, or T)(C or T)(G or C)GCCTCC (A, G, or T)(C or T)(G or C)TCCCACC GCTCCATC-3′) (SEQ ID NO: 13) and SE2 (5′-CCGGTGGCCA GGAGAATTCC GTCCTTCACG GCGATGGAGC GGTGGG-3′) (SEQ ID NO. 14) were combined with Pfu buffer, dNTPs, Pfu polymerase, and sterile water to make 50 μL. After 5 PCR cycles, pGBDRXR:3Stop and BP were added to the reaction and the PCR was continued for 30 cycles. The product (240 bp) was purified using the Zymoclean Gel DNA Recovery Kit.

SEAM was constructed in a similar way to BPSE. SE1 and SE2 were mixed with Pfu Buffer, dNTPs, Pfu polymerase, and sterile water to 25 μL. Simultaneously, AM1 (5′-GGCTCTGCGC TCCATCGGGC TTAAGTGCCT GGAACAT(A, G, or T)(C or T)(G or C) TTSCTTCTTC AAGCTCATCG GGG-3′) (SEQ ID NO. 15) and AM2 (5′-GCATCTCAAT AAGGAAGGTG TCAATTGTGT GTCCCCGATG AGCTTGAAGA A-3′) (SEQ ID NO. 16) were combined with Pfu Buffer, dNTPs, Pfu polymerase, and sterile water to 25 μL. After 5 cycles, these two reactions were mixed and pGBDRXR:3Stop was added. The PCR was continued for 30 cycles. The PCR product (460 bp) was purified using the Zymoclean Gel DNA Recovery Kit.

The AMR cassette was made similarly to FBP. AM1 and AM2 were mixed with TE containing 100 mM NaCl to make 50 μL, heated to 95° C. for 1 minute, then slowly cooled to 10° C. The annealed mixture was combined with EcoPol Buffer, dNTPs, Klenow, and sterile water to 200 μL, and kept at 25° C. for 45 min before heat inactivation at 75° C. for 20 minutes. The product (AM) was precipitated with isopropanol. Next, AM and R were combined with Pfu buffer, pGBDRXR:3Stop, dNTPs, Pfu Polymerase, and sterile water to make 50 μL for a PCR. The product (140 bp) was purified using the Zymoclean Gel DNA Recovery Kit.

The four cassettes (FBP, BPSE, SEAM, and AMR) were combined in a PCR to make the library of randomized insert cassettes (6mutlC). The library was cleaned using Bio-Spin 30 columns (Bio-Rad Laboratories, Hercules, Calif.).

Yeast selection plates and transformation. Synthetic complete (SC) media and plates were made as previously described (7). Selective plates were made without tryptophan (-Trp) and leucine (-Leu) or without adenine (-Ade), tryptophan (-Trp) and leucine (-Leu). Ligands were added to the media after cooling to 50° C.

The randomized cassette library was homologously recombined into the pGBDRXR:3Stop plasmid using the following method. pGBDRXR:3Stop was first digested with BssHll and Eagl (NEB, Beverly, Mass.), and then treated with calf intestinal phosphatase (NEB, Beverly, Mass.), to make a vector cassette. Vector cassette (1 μg) and 6mutlC (9 μg) were transformed according to Geitz's transformation protocol (8) on a 10× scale into the PJ69-4A yeast strain, which had previously been transformed with a plasmid (pGAD10BAACTR) (manuscript submitted) expressing the nuclear receptor coactivator ACTR fused to the yeast Gal4 activation domain. Homologous regions between the vector cassette and the insert cassette allow the yeast to homologously recombine the insert cassette with the vector cassette forming a circular plasmid with a complete RXR LBD gene. The transformation mixture (1 mL) was spread on each of 10 large plates of SC-Ade-Trp-Leu media containing 10 μM LG335. The transformation mixture (2 and 20 μL) was also spread on SC-Trp-Leu media. These plates were grown for 4 days at 30° C.

Molecular Modeling. Docking of LG335 in to modified binding pockets was done using the InsightII module Affinity. The wild type RXR with 9cRA crystal structure (9) was modified using the Biopolymer module residue replace tool to make mutations in the binding pocket that corresponded to the mutations in variants I268; I130A; F313A; L436F, I268V; A272V; I310L; F313M, and 1268A; I310S; F313A; L436F. The ligand was placed in the binding pocket by superimposing the carboxylate carbon and two carbons in the tetrahydronapthalene ring of LG335 onto corresponding carbons of 9cRA in the crystal structure. A Monte Carlo simulation was performed first, followed by Simulated Annealing of the best docked conformations.

Library Evaluation

To evaluate the efficiency of library creation and selection we take a binary approach—either the sequence is or is not a designed sequence. Eq. 1 is the relevant binomial distribution for statistical evaluation of the libraries.

$$P = \frac{( N - 1 ) !}{( k - 1 ) ! ( N - k ) !} {{p}^{k} ( 1 - p )}^{N - k} \qquad ( 1 )$$

In Eq. 1 N is the number of sequenced plasmids; k is the number of background or designed plasmids; p is the frequency of the occurrence of either background or designed plasmid; and P is the measure of certainty. Applying Eq. 1 to the libraries, we conclude with 95% certainty that the unselected library is at least 72% background and the selected library is at least 78% designed sequences.

Genotype Determination. Plasmids were rescued using either the Powers method (www.fhcrc.org/labs/gottschling/yeast/yplas.html) or the Zymoprep Kit (Zymo Research, Orange, Calif.). The plasmids were then transformed into Z-competent (Zymo Research, Orange, Calif.) XL 1-Blue cells (Stratagene, La Jolla, Calif.). The QIAprep Spin Miniprep Kit (Qiagen, Valencia, Calif.) was used to purify the DNA from the transformants. These plasmids were sequenced.

Quantitation Assays

Solid Media. The rescued plasmids were transformed into PJ69-4A containing the pGAD10BAACTR plasmid and plated on (SC)-Trp-Leu media. These plates were grown for 2 days at 30° C.

Colonies were streaked onto the following media: SC, SC-Trp-Leu, SC-Ade-Trp-Leu, SC-Ade-Trp-Leu plus increasing concentration of LG335 or 9cRA from 1 nM to 10 μM.

Liquid Media. The method used for quantitation was modified from a method developed by Miller and known in the art.

Mammalian Luciferase Assay. Performed with HEK 293 cells as previously described, and known in the art.

Streaking Cells onto Adenine Selective Plates Using PJ69-4A.

Yeast transformants containing the plasmids were streaked onto the selective plates (SC-Ade) with different ligand concentrations using sterile toothpicks. Plates were divided into sectors for the samples and controls; the control sectors contain pGBDMT and pGBT9Gal4. The same colony was used for streaking on all the plates, ending with a SC plate to confirm efficient transfer of the cells to each plate. Both selective and non-selective plates were incubated at 30° C. for two days. Each set of genetic selection plates was replicated at least once.

Streaking cells onto FOA plates using MaVW3

Yeast transformants containing the plasmids were streaked onto selective plates, SC-Leu-Trp, containing 5-fluororotic acid, FOA, and different ligand concentrations. Plates were also divided into sectors, with pGBT9Gal4 and pGBDMT as controls. The same procedure was used for streaking as for the adenine selection plates. Plates were incubated for two days. Each set of the genetic selection plates was replicated at least once.

### EXAMPLES

### Example 1

### Library Design

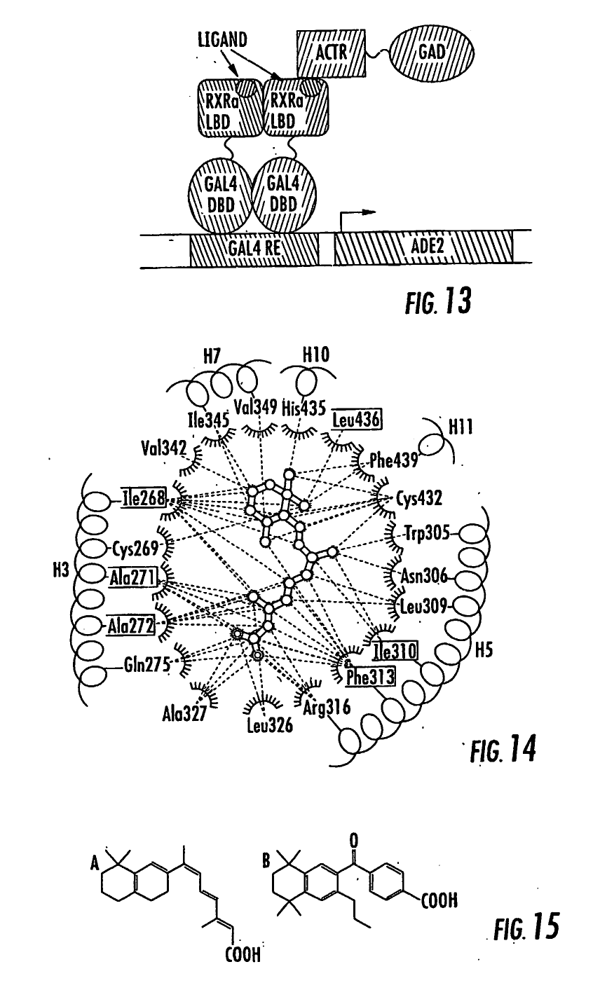

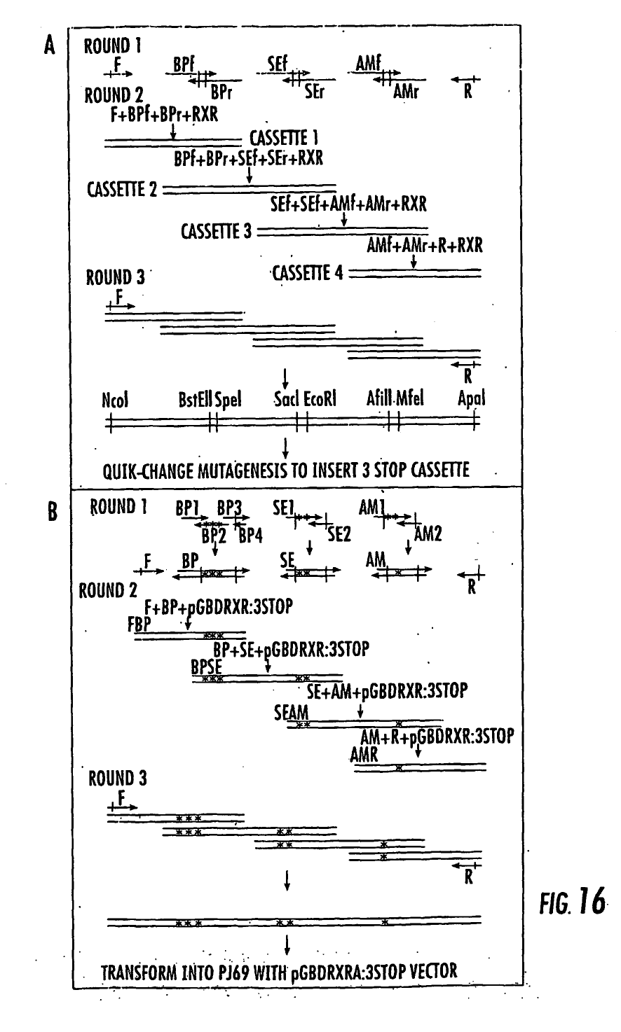

The binding pocket of the RXR LBD is composed of primarily hydrophobic side chains plus several positively charged residues that stabilize the negatively charged carboxylate group of 9cRA. The target ligand, LG335, contains an analogous carboxylate group, so the positively charged residues were left unchanged. We hypothesized that binding affinity arises from hydrophobic contacts and that specificity arises from binding pocket size, shape, hydrogen bonding, and electrostatics. The randomized amino acids were chosen based on their proximity to the bound 9cRA as observed in the crystal structure and the results of site directed mutagenesis (supporting information FIG. 14). The electrostatic interactions were held constant while the size, shape, and potential hydrogen bonding interactions were varied to find optimum contacts for LG335 binding. A library of RXRs with mutations at six positions was created. At three of the positions (I268, A271, and A272) are four possible amino acids (L, V, A, and P) and at the other three positions (I310, F313, and L436) there are eight possible amino acids (L, I, V, F, M, S, A, and T). The combination of six positions and number of encoded amino acids allowed testing of the library construction while keeping the library size (32,768 amino acid combinations and ˜3 million codon combinations) within reasonable limits. Proline was included in the library as a negative control. Residues 268, 271, and 272 are in the middle of helix 3, which would be disrupted by the inclusion of proline. Therefore, proline residues should appear at these positions only in unselected variants and not in the variants that activate in response to ligand. The substitutions at positions 268, 271, and 272 were restricted to small amino acids allowing access to the positively charged residues at this end of the pocket.

To eliminate contamination of the library with unmutated, wild-type RXR the gene was modified to create a non-functional gene, RXR:3Stop. Forty base pairs were deleted at three separate sites producing three stop codons in the coding region to create this nonfunctional gene. The deletions correspond to regions in the RXR gene where randomized codons are designed. This plasmid, pGBDRXR:3Stop, was cotransformed into yeast with the library of insert cassettes containing full-length RXR LBD genes with randomized codons at positions 268, 271, 272, 310, 313, and 436. The insert cassettes and the plasmid contain homologous regions enabling the yeast to homologously recombine the cassette into the plasmid. Recombination repairs the deletions in the RXR:3Stop gene to make full-length genes with mutations at the six specific sites.

### Example 2

### Library Selection

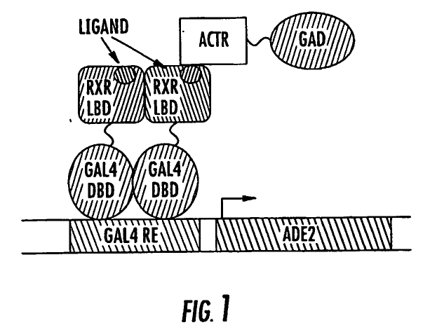

To limit the number of variants to be screened, the library was subjected to chemical complementation (FIG. 1). Chemical complementation exploits the power of genetic selection to make the survival of yeast dependent on the presence of a small molecule. The PJ69-4A strain of S. cerevisiae has been engineered for use in yeast two-hybrid genetic selection and screening assays. For selection, PJ69-4A contains the ADE2 gene under the control of a Gal4 response element. Plasmids created through homologous recombination in PJ69-4A express the Gal4 DBD fused with a variant RXR LBD (GBD:RXR). A plasmid expressing ACTR, a nuclear receptor coactivator, fused with the Gal4 activation domain (ACTR:GAD), was also transformed into PJ69-4A. If a ligand causes a variant RXR LBD to associate with ACTR, transcription of the ADE2 gene is activated. Expression of ADE2 permits adenine biosynthesis and therefore, yeast survival on media lacking adenine.

A small amount of the yeast library was plated onto media (SC-Leu-Trp) selecting only for the presence of the plasmids pGAD1 OBAACTR (expressing ACTR:GAD and containing a leucine selective marker) and mutant pGBDRXR (expressing variant GBD:RXR and containing a tryptophan selective marker). The majority of the yeast cells transformed with the RXR library were plated directly onto SC-Leu-Trp-Ade media containing 10 μM LG335, selecting for adenine production in response to the compound LG335. The transformation efficiency of this library into yeast strain PJ69-4A was 3.8×10⁴ colonies per μg DNA. This number includes both the efficiency of transforming the DNA into the cells and the homologous recombination efficiency. Of the approximately 380,000 transformants, approximately 300 grew on SC-Ade-Trp-Leu+10 μM LG335 selective media.

### Example 3

### Library Characterization

Twenty-one plasmids were rescued from yeast colonies: nine from non-selective plates (SC-Trp-Leu) and twelve from selective plates (SC-Ade-Trp-Leu+10 μM LG335). The relevant portion of plasmid DNA from these colonies was sequenced to determine the genotype (Table 1). All nine of the plasmid sequences from the non-selective plates contained at least one deletion and are non-functional genes. Of the twelve plasmids that grew on the selective media, all contain full-length RXR LBDs with designed mutations. With 95% certainty, we conclude that the unselected library is at least 72% background and the selected library is at least 78% designed sequences (supporting information).

TABLE 1

Genotypes of mutants from unselected and
selected libraries

| | | | | | | |
|---|---|---|---|---|---|---|
| Mutant | I268 | A271 | A272 | I310 | F313 | L436 |

Unselected library

| | | | | | | |
|---|---|---|---|---|---|---|
| 1 | Deleted | Deleted | Deleted | Deleted | Deleted | Deleted |
| 2 | Deleted | Deleted | Deleted | Deleted | Deleted | Deleted |
| 3 | GTA(V) | CCT(P) | CCT(P) | TCG(S) | TCG(S) | Deleted |
| 4 | Deleted | Deleted | Deleted | Deleted | Deleted | Deleted |
| 5 | Deleted | Deleted | Deleted | Deleted | Deleted | GCG(A) |
| 6 | Deleted | Deleted | Deleted | Deleted | Deleted | Deleted |
| 7 | Deleted | Deleted | Deleted | Deleted | Deleted | Deleted |
| 8 | Deleted | Deleted | Deleted | Deleted | Deleted | Deleted |
| 9 | Deleted | Deleted | Deleted | Deleted | Deleted | TTC(F) |

Selected library

| | | | | | | |
|---|---|---|---|---|---|---|
| 1 | GTG(V) | wtRXR | GCA | TTG(L) | ATG(M) | TTG |
| 2 | GTG(V) | wtRXR | GCA | GTG(V) | TCC(S) | TTG |
| 3 | CTA(L) | GCT | GCA | ATG(M) | GTG(V) | TTG |
| 4 | GCG(A) | wtRXR | GCA | TCC(S) | GTG(V) | TTC(F) |
| 5 | GCT(A) | GCT | GCA | GCC(A) | GCG(A) | TTC(F) |
| 6 | GCT(A) | GCT | GTT(V) | GCC(A) | GCG(A) | TTC(F) |
| 7 | CTT(L) | GCT | GCT | GTC(V) | ATC(I) | TTG |
| 8 | CTG(L) | GTG(V) | GCG | TTG(L) | TTG(L) | TTG |
| 9 | GTG(V) | GTG(V) | GCG | TTG(L) | GTG(V) | TTG |
| 10 | GTA(V) | wtRXR | GTG(V) | ATG(M) | TCC(S) | ATG(M) |
| 11 | GCG(A) | GCG | GCA | ATG(M) | GCG(A) | ACG(T) |
| 12 | GCG(A) | GCT | GCG | TCG(S) | GTC(A) | TTC(F) |

Sequences condons are followed by the encoded amino acid in parentheses.
“wtRXR” indicates that the sequence corresponds to the wild-type RXR condon.
“Deleted” indicates the presence of an unmutated 35top deletion background cassette.

### Example 4

### Variant Characterization in Yeast

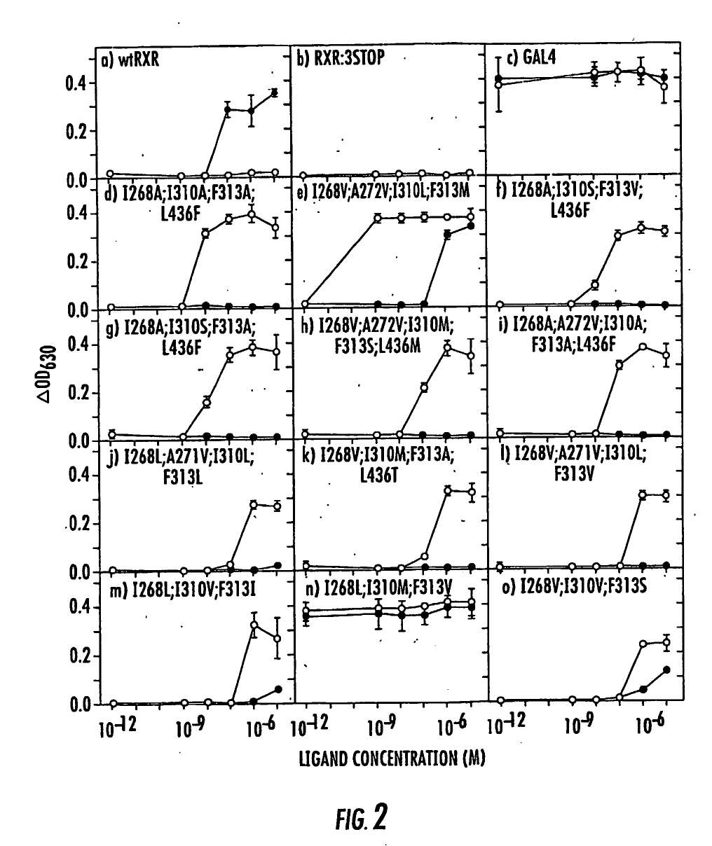

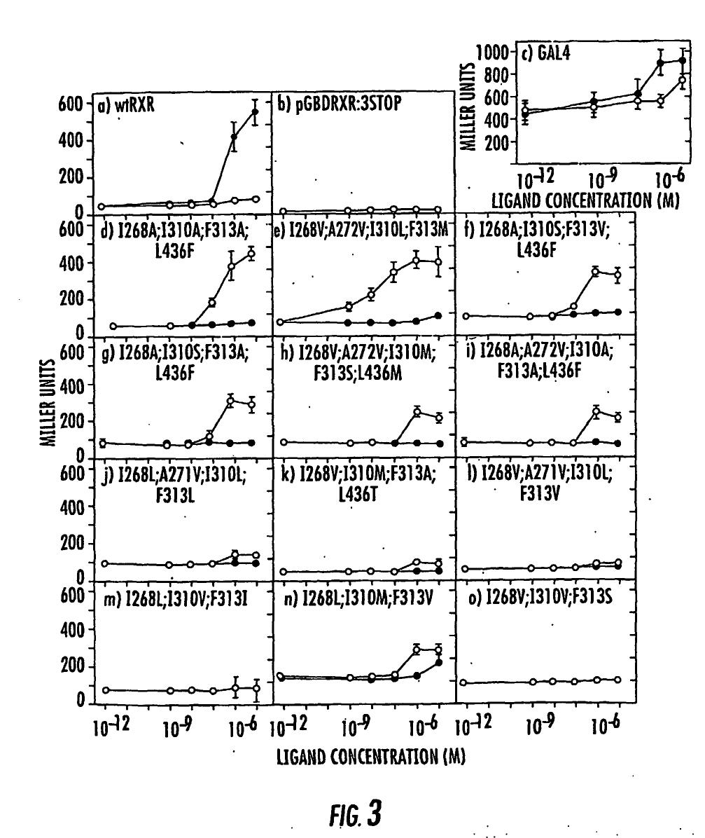

The twelve plasmids rescued from the selective plates were retransformed into PJ69-4A to confirm that their phenotype is plasmid linked. The strain PJ69-4A was engineered to contain a Gal4 response element controlling expression of the LacZ gene, in addition to the ADE2 gene. Both selection and screening were used to determine the activation level of each variant by 9cRA and LG335. The selection assay quantifies yeast growth occurring through transcriptional activation of the ADE2 gene, while the screen quantifies β-galactosidase activity occurring though transcriptional activation of the LacZ gene. Although the selection assay (FIG. 2) is ˜10-fold more sensitive than the screen (FIG. 3), it does not quantify activation level (efficacy) as well as the screen. In the selection assay, there is either growth or no growth, whereas the screen more accurately quantifies different activation levels at various concentration of ligand (FIGS. 2 and 3). The differences will be more fully discussed in a future publication.

Three plasmids were used as controls in the screen and selection assays. The plasmids pGBDRXRα and pGBT9Gal4 were used as positive controls to which the activation level of the variants can be compared. pGBDRXRα expresses the gene for the “wild-type” GBD:RXR, which grows and is activated by 9cRA but not by LG335. pGBT9Gal4 expresses the gene for the ligand-independent yeast transcription factor Gal4 (25), which is constitutively active in the presence or absence of either ligand. The plasmid pGBDRXR:3Stop serves as a negative control. pGBDRXR:3Stop carries a non-functional RXR LBD gene; therefore, yeast transformed with this plasmid does not grow in the selection assay nor show activity in the screen. This plasmid provides a measure of background noise in both the selection and screen assays.

Both the selection and screen assays show that ten of the twelve variants are selectively activated by LG335. Results of these assays are shown in FIGS. 2 and 3. Table 2 summarizes the transcriptional activation profiles of all twelve variants in response to both 9cRA and LG335 compared to wild-type RXR.

TABLE 2
EC₅₀ and efficacy in yeast and HEK 293 cells for RXR variants

| | 9CRA | | | | LG335 | | | |
|---|---|---|---|---|---|---|---|---|
| | Yeast | | HEK 293 | | Yeast | | HEK 293 | |
| Variant | EC₅₀ | Eff | EC₅₀ | Eff | EC₅₀ | Eff | EC₅₀ | Eff |
| WT | 500 | 100 | 220 | 100 | >10,000 | 10 | 300 | 10 |
| I268A; I310A; F313A; | >10,000 | 0 | >10,000 | 0 | 220 | 70 | 30 | 50 |
| L436F | | | | | | | | |
| I268V; A272V; I310L; F313M | >10,000 | 10 | 1,600 | 30 | 40 | 60 | 1 | 30 |
| I268A; I310S; F313V; L436F | >10,000 | 10 | — | — | 470 | 60 | — | — |
| I268A; I310S; F313V; L436F | >10,000 | 0 | >10,000 | 0 | 430 | 50 | 690 | 20 |
| I268V; A272V; I310M; F313S; | >10,000 | 10 | >10,000 | 0 | 680 | 30 | 180 | 30 |
| L436M | | | | | | | | |
| I268A; A272V; I310A; F313A; | >10,000 | 0 | — | — | 530 | 30 | 1 | — |
| L436F | | | | | | | | |
| I268L; A271V; I310L; F313L | >10,000 | 0 | — | — | 530 | 20 | 1 | — |
| I268A; 1310M; F313A; L436T | >10,000 | 0 | >10,000 | 0 | 610 | 10 | 140 | 20 |
| I268V; A271V; I310L; F313V | >10,000 | 0 | — | — | 650 | 10 | — | — |
| I268L; I310V; F313I | >10,000 | 0 | — | — | >2000 | 10 | — | — |
| I268L; I310M; F313V | >10,000 | 20 | — | — | 610 | 20 | — | — |
| I268V; I310V; F313S | >10,000 | 0 | — | — | 440 | 10 | — | — |

EC₅₀ values (given in nm) represent the averages of two screen experiments in quadruplicate for yeast and in triplicate for HEK 293. Efficacy (Eff; given as a percent) is the maximum increase in activation relative to the increase in activation of wild type with 10 μM 9cRA. Values represent the averages of two screen experiments in quadruplicate for yeast and in triplicate in HEK 293.

Five variants were chosen for testing in mammalian cell culture for comparison of the activation profiles (I268A; I310A; F313A; L436F, I268V; A272V; I310L; F313M, I268A; I310S; F313A; L436F, I268V; A272V; I310M; F313S; L436M, and I286A; I310M; F313A; L436T). The genes for these variants were removed from yeast expression plasmids and ligated into mammalian expression plasmids.

Although I268L; I310M; F313V is constitutively active in the selection assay (FIG. 2n) and has high basal activity in the screen assay, both 9cRA and LG335 increase activity at micromolar concentrations (FIG. 3n). This variant may be in an intermediate conformation, with weakly activated transcription that can be improved by ligand binding. The high basal activation could also be due to a change in the conformation equilibrium with a shift towards the active conformation when ligand is not present.

I268V; I310V; F313S is constitutively active on solid media (data not shown), but shows no activation in the screen (0% Eff., Table 2, FIG. 3o) and only grows in the liquid media selection after two days (FIG. 2o). The basal activation level may be below the threshold of detection for the liquid media assays. However, it is also possible that agar, which is not present in the liquid assays, contains some small molecule that activates the receptor.

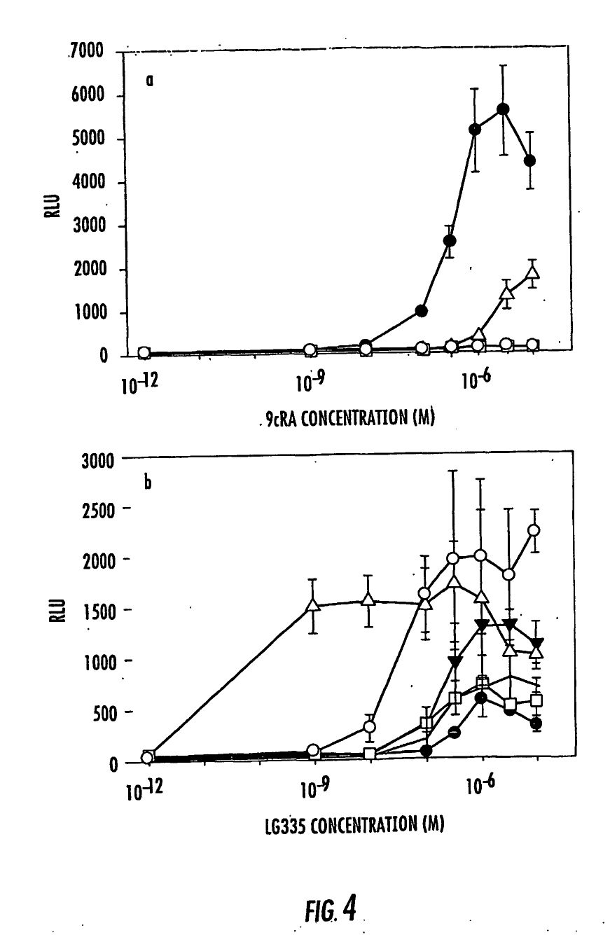

Activation levels and EC₅₀s correlate in yeast and HEK 293 cells (FIG. 4 and Table 2). For the majority of the variants 9cRA shows little or no activation in yeast or mammalian cells. Variant I268V; A272V; I310L; F313M is activated slightly by 9cRA in yeast, but in mammalian cells is activated to the same level as with both 9cRA and LG335 (FIGS. 2, 3 and 4). With one exception, all variants tested have EC₅₀s within 10-fold in yeast and mammalian cells. However, the EC₅₀s in mammalian cells are generally lower than in yeast. We speculate that this shift is due to increased penetration of LG335 into mammalian cells versus yeast.

Subtle differences in binding pocket shape can have a drastic effect on specificity. For example, the I268V; A272V; I310L; F313M variant is activated to high levels by LG335 (60% Eff. Table 2), and is only slightly activated by 10 μM 9cRA in yeast (FIG. 3e), yet the amino acid changes are extremely conservative. The volume difference between phenylalanine and methionine side chains is only ˜4 Å³ and their polarity difference is minimal (hydration potentials of the methionine and phenylalanine side chains are −0.76 kcal mol⁻¹ and −1.48 kcal mol⁻¹, respectively). The other mutations redistribute methyl groups within the binding pocket, with a net difference of one methyl group (˜18 Å³). The LG335-I268V; A272V; I310L; F313M ligand receptor pair also represents a 25-fold improvement in EC₅₀ over the previous best LG335 receptor, Q275C; I310M; F313I (40 nM vs. 1 μM in yeast). The Q275C; I310M; F313I variant was created using site directed mutagenesis. Subtle changes in the I268V; A272V; I310L; F313M variant produced a better ligand receptor pair than the Q275C; I310M; F313I variant. This conclusion is consistent with the observation that nuclear receptors bind ligands through an induced-fit mechanism. With current knowledge about protein-ligand interactions it is not possible to rationally design ligand-receptor pairs with specific activation profiles. Libraries and chemical complementation are a new way to circumvent this problem and obtain functional variants with a variety of activation profiles.

Molecular modeling was used to generate hypotheses about the structural basis of ligand specificity for the variants discovered in the library. First, mutations to smaller or more flexible side chains at positions 310, and 313 are essential to provide space for the propyl group of LG335. All variants activated by LG335 have mutations at these two positions. Second, mutations to amino acids with larger side chains at position 436 stericly clash with the methyl group at the 9 position of 9cRA. This interaction may prevent helix 12 from closing properly and therefore prevent activation by 9cRA. The only variant significantly activated by 9cRA (I268V; A272V; I310L; F313M) does not contain a mutation at position 436. Third we hypothesize that tight packing in the binding pocket may lead to lower EC₅₀s. The docking results for I268V; A272V; I310L; F313M with LG335 show that the methionine and leucine side chains pack tightly against the propyl group of LG335, which may result in tighter binding and consequently a lower EC₅₀s.

In the absence of functional data, chemical complementation may be used to test more hypotheses about the function of particular residues than would be possible through site directed mutagenesis. By making a library of changes at a single site, additional information could be obtained about the importance of side chain size, polarity, and charge over just the traditional mutation to alanine that is often used to explore single residue importance. In the absence of structural information, it is possible to make large libraries using error prone PCR or gene shuffling. Chemical complementation could also be used to select active variants from these types of libraries.

### Example 5

### Increasing the Sensitivity of Chemical Complementation with ACTR

To increase the sensitivity of chemical complementation, an adapter protein was introduced to link the mammalian nuclear receptor function to the yeast transcription apparatus, thereby overcoming the evolutionary divergence between mammalian cells and yeast. The human nuclear receptor coactivator ACTR was fused to the yeast Gal4 activation domain This plasmid, pGAD10BAACTR, expresses the ACTR:GAD fusion protein and contains a leucine marker. This plasmid was co-transformed into yeast with the plasmid pGBDRXR, which expresses the Gal4 DNA binding domain (DBD) fused to the RXR ligand binding domain (GBD:RXR) and contains a tryptophan marker. Transformants were selected on SC-Leu-Trp plates, and were streaked onto adenine selective plates (SC-Ade) containing 10⁻⁵ M 9cRA, a known ligand for RXR (FIG. 5G). Yeast containing just the pGBDRXR plasmid, the pGAD10BAACTR plasmid, a plasmid with just the Gal4 DBD (pGBDMT), and a plasmid containing the Gal4 holo protein (pGBT9Gal4) were also streaked onto these plates as controls.

After two days of incubation, growth occurs on the sector of the plate containing ACTR:GAD with GBD:RXR and on the sector of the plate with Gal4; whereas no growth occurs on the sector of the plate with GBD:RXR alone (FIG. 5G). The growth density produced by GBD:RXR and ACTR:GAD is the same as the growth produced by the holo Gal4. Importantly, GBD:RXR and ACTR:GAD produced no growth on plates without 9cRA.

Previous findings showed no growth was observed with RXR at 9cRA concentrations lower than 10⁻⁵M. To determine if the sensitivity of our system had increased with the introduction of the adapter fusion protein, a dose response was performed on adenine selective plates (SC -Ade) containing ligand concentrations ranging from 10⁻⁵M to 10⁻⁹M. After two days of incubation, a clear dose response occurs on the plates (FIG. 5). Without ligand, growth occurs only on the Gal4 sector of the plate, as expected At concentrations as low as 10⁻⁸ M 9cRA, ligand-activated growth occurs only on the sector of the plate containing both GBD:RXR with ACTR:GAD (FIG. 5D). At concentrations of ligand above 10⁻⁸ M, higher density growth is observed on the sector of the plate containing GBD:RXR with ACTR:GAD. No growth occurs with GBD:RXR alone as expected. In summary, the introduction of the fusion protein ACTR:GAD increases the sensitivity of chemical complementation. Growth occurs on adenine selective plates with 9cRA after two days of incubation (FIG. 5). Ligand-activated growth is observed at 9cRA concentrations as low as 10⁻⁸ M 9cRA. With chemical complementation, an approximate EC₅₀ value between 10⁻⁸ M and 10⁻⁷ M for wild-type RXR and 9cRA, which is comparable to the EC₅₀ value measured for wild-type RXR in mammalian cell assays (˜10⁻⁷ M) (FIG. 5). The growth density and rate with the ACTR:GAD fusion protein is comparable to Gal4 activated growth. The same results were obtained on adenine selective plates (SC-Ade-Trp and SC-Ade-Leu-Trp) and on histidine selective plates (data not shown). In summary, introducing an adapter fusion protein of the human coactivator with the Gal4 activation domain increases the sensitivity of chemical complementation 1000-fold, making this system more efficient for analysis of protein/ligand interactions.

### Example 6

### Increasing Sensitivity of Chemical Complementation using SRC-1

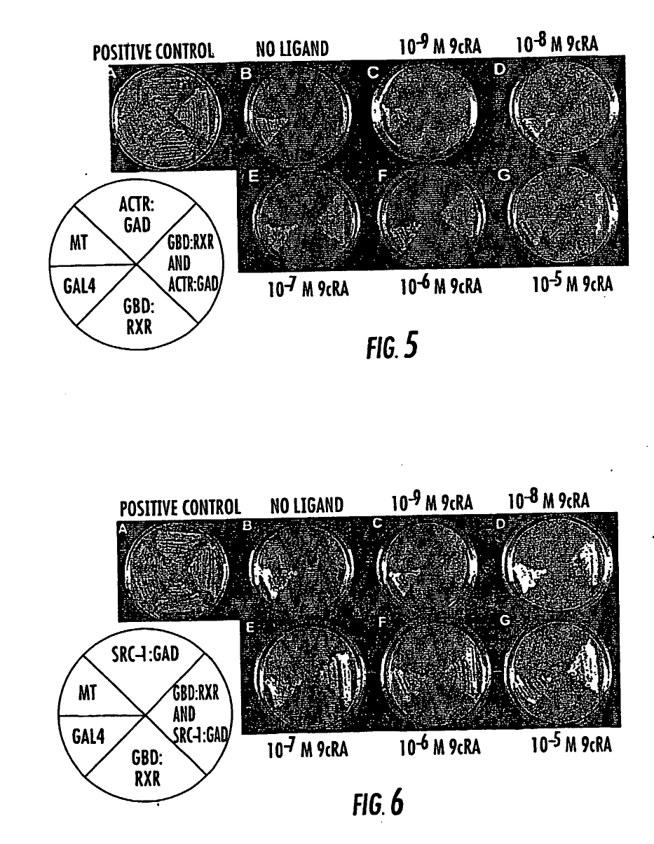

Another RXR coactivator was tested to increase the sensitivity of chemical complementation. Residues 54 to 1442 of the human nuclear receptor coactivator, SRC-1, were fused to the Gal4 activation domain to construct the plasmid pGAD10BASRC1. This plasmid, which expresses SRC1:GAD in yeast and contains a leucine marker was transformed with GBD:RXR; transformants selected from SC-Leu-Trp were streaked onto adenine selective plates (SC-Ade) with various concentrations of 9cRA (FIG. 6). Ligand-activated growth is observed only in the sector of the plate containing both GBD:RXR with SRC1:GAD, and the same trend is observed with SRC-I as the ACTR coactivator (FIG. 6).

To verify that the increased sensitivity is from specific interactions between the coactivator and the active conformation of the receptor, a series of further controls was devised. pGAD10, a plasmid containing the Gal4 activation domain (GAD) without a coactivator domain was cotransformed with pGBDRXR. The plasmid was also transformed alone. pGAD10BAACTR, pGAD10BASRC1, pGBT9Gal4, and pGBDMT were all transformed individually. These controls were streaked onto adenine selective plates (SC-Ade) with and without 9cRA.0 In the absence of ligand, only the entire Gal4 gene (pGBT9Gal4) grows as expected (data not shown). In the presence of 10⁻⁵ M 9cRA, growth occurs with the GBD:RXR with ACTR:GAD and GBD:RXR with SRC1:GAD. The Gal4 AD only (without the coactivator domain) with GBD:RXR displays no growth. These results verify that the increase in chemical complementation is specifically due to the interaction of the coactivator fusion protein with the ligand-bound nuclear receptor (data not shown).

### Example 7

### Chemical Complementation and Negative Selection

Negative selection is the opposite of classical genetic complementation. Instead of allowing the microbe to survive, a functional gene kills the microbe; only cells containing non-functional genes survive and form colonies on selective plates. Negative selection is useful for finding mutations that disrupt the function of a protein.

For negative selection in yeast, others have generated yeast strains that contain Gal4 response elements (REs) fused to the URA3 gene. The URA3 gene codes for or orotidine-5′-phosphate decarboxylase, an enzyme in the uracil biosynthetic pathway. This gene can be used for both positive and negative selection. For positive selection, yeast expressing this gene will survive in the absence of uracil in the media. For negative selection, uracil and 5-fluoroorotic acid (FOA) is added to the media. Expression of orotidine-5′-phosphate decarboxylase coverts FOA to the toxin 5-fluorouracil, which kills the yeast. As used herein, the term “negative chemical complementation” refers to negative selection that occurs due to the presence of a small molecule.

Plasmids pGBDRXR and pGAD10BAACTR were individually transformed and co-transformed into MaV103. Transformants were streaked onto uracil selective plates (SC-Ura-Trp) with 9cRA for positive selection (data not shown). The same trend was seen with the ACTR:GAD with GBD:RXR in the MaV103 strain as seen previously with the PJ69-4A strain. The same transformants were streaked onto selective plates (SC-Leu-Trp) with FOA for negative chemical complementation. Varying concentrations of 9cRA were also added to the plates, ranging from 10⁻⁵ M to 10⁻⁸ M. In the absence of ligand (FIG. 7B), yeast grow on the sector of the plate containing ACTR:GAD with GBD:RXR as expected. This is expected because uracil is provided, and in the absence of ligand RXR maintains its inactive conformation, preventing ACTR:GAD from binding and transcription does not occur. Without expression of the URA3 gene, 5-fluorouracil is not produced and the yeast survive. However, as the concentration of ligand increases (FIG. 7B-7F), less growth occurs and at the highest concentration of ligand, 10⁻⁵ M, very little growth occurs. The small amount of growth that is observed is due to background growth associated with negative selection in this strain.

Negative chemical complementation is advantageous for engineering receptors for new small molecules for several reasons. First, mutant receptor libraries may contain constitutively active receptors or receptors that activate transcription in response to endogenous small molecules. These undesirable receptors can be removed from the library with negative selection. Second, in some cases it will be desirable to remove members of the library that activate in response to certain small molecules, e.g. the natural ligands. Negative chemical complementation will remove these members of the library. The remaining library can then be put through chemical complementation with the small molecule of interest. Third, for enzyme engineering negative chemical complementation can remove library members that produce a particular small molecule, e.g. an enantiomer of the compound of interest. The remaining mutant enzyme library can then be put through chemical complementation to find those capable of producing the small molecule of interest. Fourth, for drug discovery, chemical libraries can be efficiently evaluated for antagonists of nuclear receptors by their ability to allow the yeast to survive negative chemical complementation.

### Example 8

### Chemical Complementation with RXR Mutants

Several RXR mutants previously tested in both mammalian cell assays and with chemical complementation in yeast (without the coactivator fusion protein) showed a general, but less than complete correlation. Without the coactivator fusion protein, ligand-activated growth was observed only with wild-type RXR and the F439L mutant after five days of incubation; none of the other mutants showed ligand-activated growth. The variation in the transcription machinery could lead to the different patterns in activation. To test whether the adapter fusion protein could overcome the differences and show a more direct correlation, all the mutants in Table 3 were cloned into pGBD vectors and cotransformed into yeast with pGAD10BAACTR. Again, transformants were selected from SC-Leu-Trp plates and then streaked onto adenine selective plates (SC-Ade-Trp). These mutants were tested with 9cRA and LG335 (a near-drug, a synthetic compound structurally similar to an RXR agonist but that does not activate wild-type RXR) (Table 3).

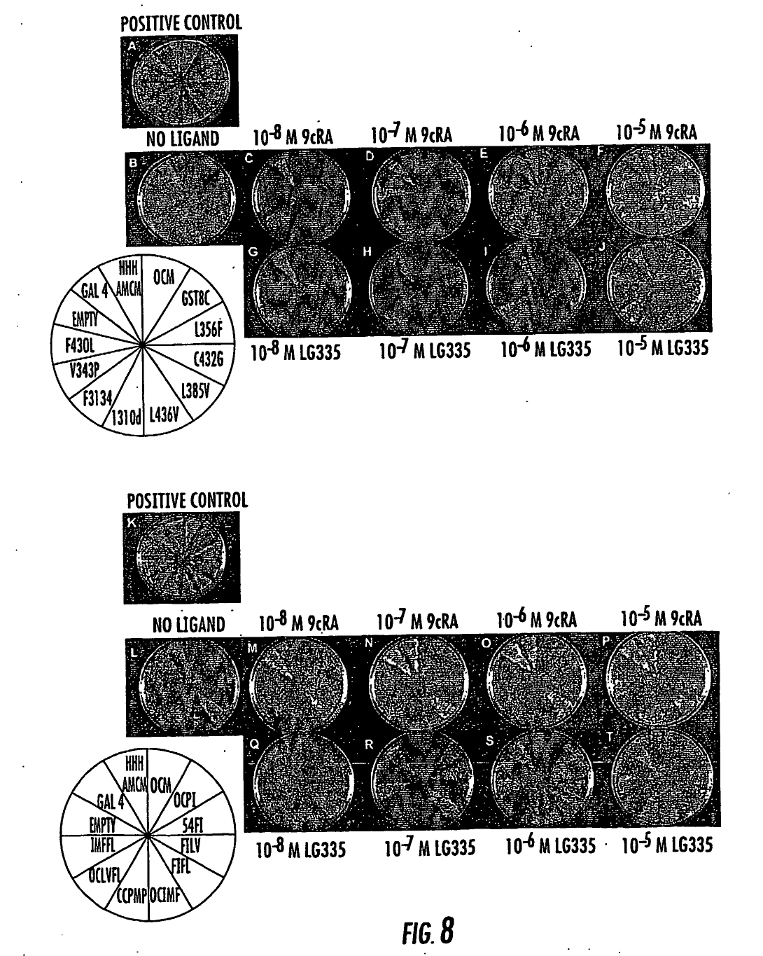

The transcriptional activation patterns of these mutants in chemical complementation with the addition of ACTR:GAD was observed on dose response plates containing both 9cRA and the synthetic ligand, LG335 (FIG. 8). On the plate without ligand, growth occurs on the sector of the plate containing Gal4, but growth also occurs on the sector of the plate with the two mutants F313I and F313I; F439L, This could be a result of the mutations causing a structural modification to the binding pocket that is favorable for the binding of an endogenous small molecule in yeast. At 10⁻⁵M 9cRA, growth occurs on the sectors of the plate with the single mutants, C432G, Q275C, I268F, I310M, V342F, and F439L, as well as some of the triple mutants I310M; F313I; F439L and Q275C; F313I; V342F. As the concentration of ligand decreases, some mutants no longer show ligand-activated growth. At 10⁻⁷ M 9cRA, growth is observed with the F439L mutant as well as wild-type RXR (FIG. 8). At the lowest concentration of ligand, 10⁻⁸ M 9cRA, growth is observed in the Gal4 and F313I sectors of the plates. For the synthetic ligand LG335, growth is observed with several of the single, double and triple mutants at 10⁻⁵ M (FIG. 8). At lower concentrations of ligand, the single mutants do not show much growth. However, several of the double and triple mutants I310M; F313I; F439L, Q275C; F313I, and I310M; F313I display ligand-activated growth at 10⁻⁷M LG335. At 10⁻⁸ M LG335, some growth is still observed in the I310M; F313I; F439L sector of the plate.

A correlation is apparent between yeast growth and transcriptional activation in mammalian cells when quantitating these results and comparing them with results from cell culture assays (Table 3). The I268F, Q275C, C432G, I310M, and I310M; F313I; F439L mutations which had previously not shown any growth with chemical complementation, grow with the ACTR:GAD fusion protein (FIG. 8). The more direct correlation between chemical complementation and mammalian cell assays shows that the coactivator fusion protein (ACTR:GAD) serves to bridge millions of years of evolution by adapting mammalian nuclear receptor function to the yeast transcription machinery.

Definitions

As used herein, the term “polynucleotide” generally refers to any polyribonucleotide or polydeoxyribonucleotide, which may be unmodified RNA or DNA or modified RNA or DNA. Thus, for instance, polynucleotides as used herein refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. The terms “nucleic acid,” “nucleic acid sequence,” or “oligonucleotide” also encompasses a polynucleotide as defined above.

In addition, polynucleotide as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide.

It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including simple and complex cells, inter alia.

The term “oligonucleotide” refers to relatively short polynucleotides. Typically the term refers to single-stranded deoxyribonucleotides, but it can refer as well to single- or double-stranded ribonucleotides, RNA:DNA hybrids and double-stranded DNAs, among other compounds containing multiple nucleotides linked through phosphodiester bonds. The phosphodiester bonds are typically 5′-3′ linkages between the deoxyribose or ribose sugars of adjacent nucleotides, which is the predominant mode of nucleotide coupling in natural DNA or RNA, respectively. The nucleotides of an oligonucleotide can be the naturally occurring ribonucleotides, rA, rC, rG and rU; deoxyribonucleotides, dA, dC, dG and dT; or other compounds in which the backbone and/or the base moieties differ from the standard nucleotides of DNA and RNA.

The term “non-natural” means not typically found in nature including those items modified by man. Non-natural includes chemically modified subunits such as nucleotides as well as biopolymers having non-natural linkages, backbones, or substitutions.

The term “non-natural backbone” means a covalent chemical linkage that couples together two or more nucleotides in a manner that is not identical to the naturally-occurring RNA or DNA phosphodiester backbones. Chemical deviations from the natural backbone can include, but are not limited to, chemical modification of a single site on the natural backbone or the replacement of a component of the backbone with a completely different chemical group. Methylation of the O2′ site on the ribose sugar is an example of a chemical difference from the natural backbone that would constitute a non-natural backbone. Replacement of the ribose sugar with a hexose sugar and/or replacement of the phosphate group in DNA or RNA with a phosphorothioate group are also examples of non-natural backbones. Exemplary modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and borano-phosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Representative oligonucleotides having inverted polarity comprise a single 3′ to 3′ linkage at the 3′-most internucleotide linkage i.e. a single inverted nucleoside residue which may be abasic (the nucleobase is missing or has a hydroxyl group in place thereof).

Some oligonucleotide backbones do not include a phosphorus atom therein and have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Some embodiments synthesize or use oligonucleotides with phosphorothioate backbones and oligonucleosides with heteroatom backbones, and in particular —CH₂—NH—O—CH₂—, —CH₂—N(CH₃)O—CH₂— [known as a methylene (methylimino) or MMI backbone], —CH₂—O—N(CH₃)—CH₂—, —CH₂—N(CH₃)—N(CH₃)—CH₂— and —O—N(CH₃)—CH₂—CH₂— [wherein the native phosphodiester backbone is represented as —O—P—O—CH₂—] of the above referenced U.S. Pat. No. 5,489,677, and the amide backbones of the above referenced U.S. Pat. No. 5,602,240.

In other embodiments, the disclosed methods and compositions may comprise modified oligonucleotides containing one or more substituted sugar moieties. Other modified oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)nON[(CH₂)ₙCH₃]₂, where n and m are from 1 to about 10. Other oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving pharmacokinetic properties and other substituents having similar properties. Another modification includes 2′-methoxyethoxy (2′-O—CH₂CH₂OCH₃, also known as 2′-O-(2-methoxyethyl) or 2′-MOE) (Martin et al. (1995) Helv. Chim. Acta, 78, 486-504) i.e., an alkoxyalkoxy group. A further preferred modification includes 2′-dimethylaminooxyethoxy, i.e., a O(CH₂)₂ON(CH₃)₂ group, also known as 2′-DMAOE, and 2′-dimethylaminoethoxyethoxy (also known in the art as 2′-O-dimethyl-amino-ethoxy-ethyl or 2′-DMAEOE), i.e., 2′-O—CH₂—O—CH₂-N(CH₃)₂.

Other modifications include 2′-methoxy (2′-O—CH₃), 2′-aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. An exemplary 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar.

A further modification includes Locked Nucleic Acids (LNAs) in which the 2′-hydroxyl group is linked to the 3′ or 4′ carbon atom of the sugar ring thereby forming a bicyclic sugar moiety. The linkage is preferably a methelyne (—CH₂—)ₙ group bridging the 2′ oxygen atom and the 4′ carbon atom wherein n is 1 or 2. LNAs and preparation thereof are described in U.S. Pat. No. 6,268,490 and WO 99/14226.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g., 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia of Polymer Science and Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases may be particularly useful for increasing the binding affinity of the oligomeric compounds of the disclosure. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2.degree. C. (Sanghvi, Y. S., Crooke, S. T. and Lebleu, B., eds., Antisense Research and Applications, CRC Press, Boca Raton, 1993, pp. 276-278) and are presently preferred base substitutions, even more particularly when combined with 2′-O-methoxyethyl sugar modifications.

The terms “including”, “such as”, “for example” and the like are intended to refer to exemplary embodiments and not to limit the scope of the present disclosure.

The term “polypeptides” includes proteins and fragments thereof. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

“Variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the structure of the polypeptides of in disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly, where the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue:exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, and 95% sequence identity to the polypeptide of interest.

“Identity,” as known in the art, is a relationship between two or more polypeptide sequences, as determined by comparing the sequences. In the art, “identity” also means the degree of sequence relatedness between polypeptide as determined by the match between strings of such sequences. “Identity” and “similarity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48:1073 (1988).

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48: 443-453, 1970) algorithm (e.g., NBLAST, and XBLAST). The default parameters are used to determine the identity for the polypeptides of the present invention.

By way of example, a polypeptide sequence may be identical to the reference sequence, that is be 100% identical, or it may include up to a certain integer number of amino acid alterations as compared to the reference sequence such that the % identity is less than 100%. Such alterations are selected from: at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the reference polypeptide by the numerical percent of the respective percent identity (divided by 100) and then subtracting that product from said total number of amino acids in the reference polypeptide.

“Operably linked” refers to a juxtaposition wherein the components are configured so as to perform their usual function. For example, control sequences or promoters operably linked to a coding sequence are capable of effecting the expression of the coding sequence.

As used herein, the term “transfection” refers to the introduction of a nucleic acid sequence into the interior of a membrane enclosed space of a living cell, including introduction of the nucleic acid sequence into the cytosol of a cell as well as the interior space of a mitochondria, nucleus or chloroplast. The nucleic acid may be in the form of naked DNA or RNA, associated with various proteins or the nucleic acid may be incorporated into a vector.

As used herein, the term “vector” is used in reference to a vehicle used to introduce a nucleic acid sequence into a cell. A viral vector is virus that has been modified to allow recombinant DNA sequences to be introduced into host cells or cell organelles.

The term “selective agent” refers to a substance that is required for growth or for preventing growth of a cell or microorganism, for example cells or microorganisms that have been engineered to require a specific substance for growth or inhibit or reduce growth in the absence of a complementing factor. Exemplary complementing factors include enzymes that degrade the selective agent, or enzymes that produce a selective agent. Generally, selective agents include, but are not limited to amino acids, antibiotics, nucleic acids, minerals, nutrients, etc. Selective media generally refers to culture media deficient in at least one substance, for example a selective agent, required for growth. The addition of a selective agent to selective media results in media sufficient for growth.

As used herein, the term “coregulator” refers to a transcription modulator.

It should be emphasized that the above-described embodiments of the present disclosure, particularly, any “preferred” embodiments, are merely possible examples of implementations, merely set forth for a clear understanding of the principles of the disclosed subject matter. Many variations and modifications may be made to the above-described embodiment(s) without departing substantially from the spirit and principles of the disclosure. All such modifications and variations are intended to be included herein within the scope of this disclosure and protected by the following claims.

We claim:

1. A method for identifying receptors, comprising:
(a) introducing a first polynucleotide encoding a receptor in to a cell, wherein the receptor comprises a ligand binding domain for a target ligand operably linked to a polynucleotide binding domain so that binding of the target ligand to the receptor activates transcription of a second polynucleotide complementing a selection agent;
(b) culturing the cell on the selective media in the presence of the target ligand, wherein growth of the cell indicates interaction of the receptor with the target ligand.

2. The method of claim 1, further comprising culturing the cell on selective media in the absence of the target ligand, wherein growth of the cell indicates the receptor constitutively activates transcription of the second polynucleotide.

3. A cell comprising:
(a) a recombinant nuclear receptor that induces expression of a first polynucleotide in response to interaction with a target substance, wherein expression of the first polynucleotide complements a selective agent; and
(b) an adapter fusion protein comprising a human coregulator domain operably linked to an activation domain, wherein the adapter fusion protein enhances transcription of the first polynucleotide induced by the recombinant nuclear receptor.

4. The cell of claim 3, wherein the cell is eukaryotic or prokaryotic.

5. The cell of claim 3, wherein the cell is a yeast cell.

6. A method for identifying enzymes comprising:
(a) introducing a first polynucleotide into a cell that is unable to grow on selective media, wherein the cell expresses a recombinant receptor polypeptide that activates transcription of a second polynucleotide in response to interaction of the recombinant receptor polypeptide with a target substance;
(b) culturing the cell on the selective media; and
(c) selecting the cell that grows on the selective media.

7. The method of claim 6, wherein the target substance is produced by a polypeptide encoded by the first polynucleotide.

8. The method of claim 6, wherein a single target substance induces a conformational change in the recombinant receptor to activate transcription.

9. The method of claim 6, wherein the target substance is unmodified.

10. The method of claim 6, wherein growth on the selective media indicates the first polynucleotide encodes a product that complements the selective media.

11. The method of claim 6, wherein the cell is a eukaryotic or prokaryotic cell.

12. The method of claim 6, wherein the selective media does not contain an amino acid necessary for survival.

13. The method of claim 12, wherein the amino acid is selected from the group consisting of histidine and alanine.

14. The method of claim 6, wherein the first polynucleotide encodes an enzyme that produces the target substance.

15. The method of claim 6, wherein the transformed cell further expresses an adaptor fusion protein.

16. The method of claim 7, wherein the adaptor fusion protein 16.

17. The method of claim 6, wherein the first polynucleotide encodes an engineered enzyme.

18. The method of claim 6, wherein the first polynucleotide encodes a naturally occurring enzyme.

19. The method of claim 6, comprises a human coactivator for transcription of the second polynucleotide.

20. A cell comprising:
(a) a recombinant nuclear receptor that induces transcription of a first polynucleotide in response to interaction with a target substance; and
(b) an adapter fusion protein comprising a human coactivator domain operably linked to an activation domain, wherein the adapter fusion protein enhances transcription of the first polynucleotide induced by the recombinant nuclear receptor.

21. The cell of claim 20, wherein the human coactivator domain is selected from the group consisting of SRC-1 and ACTR.

22. The cell of claim 20, wherein the cell is unable to grow on selective media.

23. A method for selecting cells comprising:
(a) introducing a first polynucleotide into a cell, wherein the cell expresses a recombinant receptor polypeptide that activates transcription of a second polynucleotide in response to interaction of the recombinant receptor polypeptide with a target substance;
(b) culturing the cell on selective media in the presence of a first selection agent; and
(c) selecting the cell that survives on the selective media in the presence of the selection agent, wherein expression of the second polynucleotide inhibits growth of the cell.

24. The method of claim 23, wherein the second polynucleotide encodes a cytotoxic polypeptide.

25. The method of claim 24, wherein the cytotoxic polypeptide comprises a proapoptotic polypeptide.

26. The method of claim 21, wherein the first selective agent comprises 5-fluoroorotic acid.

27. The method of claim 26, wherein the second polynucleotide encodes orotidine-5′-phosphate decarboxylase.

28. The method of claim 27, wherein the toxic substance comprises 5-fluorouracil.

29. A method for assembling an enzymatic pathway comprising:
(a) introducing a plurality of polynucleotides encoding enzymes having different substrates into a cell that is unable to grow on selective media, wherein the cell expresses a recombinant receptor polypeptide that activates transcription of a second polynucleotide in response to interaction of the recombinant receptor polypeptide with a target substance;
(b) culturing the cell on the selective media; and
(c) selecting the cell that grows on the selective media, wherein growth of a cell on the selective media indicates that the plurality of polynucleotides encode enzymes for producing products that complement the selective media.

30. The method of claim 31, wherein the product of one of the enzymes is the substrate of another of the enzymes.

31. A method for identifying receptors, comprising:
(a) introducing a first polynucleotide encoding a receptor in to a cell, wherein the receptor comprises a ligand binding domain for a target ligand operably linked to a response element so that binding of the target ligand to the receptor activates transcription of a second polynucleotide complementing a selection agent;
(b) culturing the cell on the selective media in the presence of the target ligand, wherein growth of the cell indicates interaction of the receptor with the target ligand.


SEQUENCE LISTING (18 sequences)

SEQ ID NO: 1 - DNA, 17 bp, artificial sequence
  primer
cggaatttcccatgggc

SEQ ID NO: 2 - DNA, 37 bp, artificial sequence
  primer
ctcgccgaacgacccggtcaccgcatgccactagtgg

SEQ ID NO: 3 - DNA, 36 bp, artificial sequence
  primer
ccgcttggcccactccactagtggcatgcggtgacc

SEQ ID NO: 4 - DNA, 37 bp, artificial sequence
  primer
cgggcaggctggaatgagctcctcgacggaattctcc

SEQ ID NO: 5 - DNA, 36 bp, artificial sequence
  primer
cagcccggtggccaggagaattccgtcgaggagctc

SEQ ID NO: 6 - DNA, 40 bp, artificial sequence
  primer
ctctgcgctccatcgggcttaagtgcccaccaattgacac

SEQ ID NO: 7 - DNA, 46 bp, artificial sequence
  primer
ctccagcatctccataaggaaggtgtcaattggtgggcacttaagc

SEQ ID NO: 8 - DNA, 17 bp, artificial sequence
  primer
caaaggatgggccgcag

SEQ ID NO: 9 - DNA, 46 bp, artificial sequence
  primer
ggcaaacatggggctgaaccccagctcgccgaacgacccggtcacc

SEQ ID NO: 10 - DNA, 66 bp, artificial sequence
  primer
  misc_feature (33)..(38): n is a, c, g, or t
  misc_feature (45)..(46): n is a, c, g, or t
gcccactccactagtgtgaaaagctgtttgtcnnnnnnttggcanngttggtgaccgggtcgttcg

SEQ ID NO: 11 - DNA, 48 bp, artificial sequence
  primer
cttttcacactagtggagtgggccaagcggatcccacacttctcagag

SEQ ID NO: 12 - DNA, 28 bp, artificial sequence
  primer
ggggcagctctgagaagtgtgggatccg

SEQ ID NO: 13 - DNA, 48 bp, artificial sequence
  primer
  misc_feature (22)..(24): n is a, c, g, or t
  misc_feature (31)..(33): n is a, c, g, or t
gcaggctggaatgagctcctcnnngcctccnnntcccaccgctccatc

SEQ ID NO: 14 - DNA, 46 bp, artificial sequence
  primer
ccggtggccaggagaattccgtccttcacggcgatggagcggtggg

SEQ ID NO: 15 - DNA, 63 bp, artificial sequence
  primer
  misc_feature (38)..(40): n is a, c, g, or t
ggctctgcgctccatcgggcttaagtgcctggaacatnnnttscttcttcaagctcatcgggg

SEQ ID NO: 16 - DNA, 51 bp, artificial sequence
  primer
gcatctcaataaggaaggtgtcaattgtgtgtccccgatgagcttgaagaa

SEQ ID NO: 17 - DNA, 12414 bp, artificial sequence
  artificial vector
gcttgcatgcaacttcttttctttttttttcttttctctctcccccgttgttgtctcaccatatccgcaatgacaaaaaaaatgatggaagacactaaaggaaaaaattaacgacaaagacagcaccaacagatgtcgttgttccagagctgatgaggggtatcttcgaacacacgaaactttttccttccttcattcacgcacactactctctaatgagcaacggtatacggccttccttccagttacttgaatttgaaataaaaaaagtttgccgctttgctatcaagtataaatagacctgcaattattaatcttttgtttcctcgtcattgttctcgttccctttcttccttgtttctttttctgcacaatatttcaagctataccaagcatacaatcaactccaagctttgcaaagatggataaagcggaattaattcccgagcctccaaaaaagaagagaaaggtcgaattgggtaccgccgccaattttaatcaaagtgggaatattgctgatagctcattgtccttcactttcactaacagtagcaacggtccgaacctcataacaactcaaacaaattctcaagcgctttcacaaccaattgcctcctctaacgttcatgataacttcatgaataatgaaatcacggctagtaaaattgatgatggtaataattcaaaaccactgtcacctggttggacggaccaaactgcgtataacgcgtttggaatcactacagggatgtttaataccactacaatggatgatgtatataactatctattcgatgatgaagataccccaccaaacccaaaaaaagagatctttatgagtggattaggagaaaacttggatccactggccagtgattcacgaaaacgcaaattgccatgtgatactccaggacaaggtcttacctgcagtggtgaaaaacggagacgggagcaggaaagtaaatatattgaagaattggctgagctgatatctgccaatcttagtgatattgacaatttcaatgtcaaaccagataaatgtgcgattttaaaggaaacagtaagacagatacgtcaaataaaagagcaaggaaaaactatttccaatgatgatgatgttcaaaaagccgatgtatcttctacagggcagggagttattgataaagactccttaggaccgcttttacttcaggcattggatggtttcctatttgtggtgaatcgagacggaaacattgtatttgtatcagaaaatgtcacacaatacctgcaatataagcaagaggacctggttaacacaagtgtttacaatatcttacatgaagaagacagaaaggattttcttaagaatttaccaaaatctacagttaatggagtttcctggacaaatgagacccaaagacaaaaaagccatacatttaattgccgtatgttgatgaaaacaccacatgatattctggaagacataaacgccagtcctgaaatgcgccagagatatgaaacaatgcagtgctttgccctgtctcagccacgagctatgatggaggaaggggaagatttgcaatcttgtatgatctgtgtggcacgccgcattactacaggagaaagaacatttccatcaaaccctgagagctttattaccagacatgatctttcaggaaaggttgtcaatatagatacaaattcactgagatcctccatgaggcctggctttgaagatataatccgaaggtgtattcagagattttttagtctaaatgatgggcagtcatggtcccagaaacgtcactatcaagaagttaccagtgatgggatattttccccaacagcttatcttaatggccatgcagaaaccccagtatatcgattctcgttggctgatggaactatagtgactgcacagacaaaaagcaaactcttccgaaatcctgtaacaaatgatcgacatggctttgtctcaacccacttccttcagagagaacagaatggatatagaccaaacccaaatcctgttggacaagggattagaccacctatggctggatgcaacagttcggtaggcggcatgagtatgtcgccaaaccaaggcttacagatgccgagcagcagggcctatggcttggcagaccctagcaccacagggcagatgagtggagctaggtatgggggttccagtaacatagcttcattgacccctgggccaggcatgcaatcaccatcttcctaccagaacaacaactataggctcaacatgagtagccccccacatgggagtcctggtcttgccccaaaccagcagaatatcatgatttctcctcgtaatcgtgggagtccaaagatagcctcacatcagttttctcctgttgcaggtgtgcactctcccatggcatcttctggcaatactgggaaccacagcttttccagcagctctctcagtgccctgcaagccatcagtgaaggtgtggggacttcccttttatctactctgtcatcaccaggccccaaattggataactctcccaatatgaatattacccaaccaagtaaagtaagcaatcaggattccaagagtcctctgggcttttattgcgaccaaaatccagtggagagttcaatgtgtcagtcaaatagcagagatcacctcagtgacaaagaaagtaaggagagcagtgttgagggggcagagaatcaaaggggtcctttggaaagcaaaggtcataaaaaattactgcagttacttacctgttcttctgatgaccggggtcattcctccttgaccaactcccccctagattcaagttgtaaagaatcttctgttagtgtcaccagcccctctggagtctcctcctctacatctggaggagtatcctctacatccaatatgcatgggtcactgttacaagagaagcaccggattttgcacaagttgctgcagaatgggaattcaccagctgaggtagccaagattactgcagaagccactgggaaagacaccagcagtataacttcttgtggggacggaaatgttgtcaagcaggagcagctaagtcctaagaagaaggagaataatgcacttcttagatacctgctggacagggatgatcctagtgatgcactctctaaagaactacagccccaagtggaaggagtggataataaaatgagtcagtgcaccagctccaccattcctagctcaagtcaagagaaagaccctaaaattaagacagagacaagtgaagagggatctggagacttggataatctagatgctattcttggtgatctgactagttctgacttttacaataattccatatcctcaaatggtagtcatctggggactaagcaacaggtgtttcaaggaactaattctctgggtttgaaaagttcacagtctgtgcagtctattcgtcctccatataaccgagcagtgtctctggatagccctgtttctgttggctcaagtcctccagtaaaaaatatcagtgctttccccatgttaccaaagcaacccatgttgggtgggaatccaagaatgatggatagtcaggaaaattatggctcaagtatgggagactggggcttaccaaactcaaaggccggcagaatggaacctatgaattcaaactccatgggaagaccaggaggagattataatacttctttacccagacctgcactgggtggctctattcccacattgcctcttcggtctaatagcataccaggtgcgagaccagtattgcaacagcagcagcagatgcttcaaatgaggcctggtgaaatccccatgggaatgggggctaatccctatggccaagcagcagcatctaaccaactgggttcctggcccgatggcatgttgtccatggaacaagtttctcatggcactcaaaataggcctcttcttaggaattccctggatgatcttgttgggccaccttccaacctggaaggccagagtgacgaaagagcattattggaccagctgcacactcttctcagcaacacagatgccacaggcctggaagaaattgacagagctttgggcattcctgaacttgtcaatcagggacaggcattagagcccaaacaggatgctttccaaggccaagaagcagcagtaatgatggatcagaaggcaggattatatggacagacatacccagcacaggggcctccaatgcaaggaggctttcatcttcagggacaatcaccatcttttaactctatgatgaatcagatgaaccagcaaggcaattttcctctccaaggaatgcacccacgagccaacatcatgagaccccggacaaacacccccaagcaacttagaatgcagcttcagcagaggctgcagggccagcagtttttgaatcagagccgacaggcacttgaattgaaaatggaaaaccctactgctggtggtgctgcggtgatgaggcctatgatgcagccccagcagggttttcttaatgctcaaatggtcgcccaacgcagcagagagctgctaagtcatcacttccgacaacagagggtggctatgatgatgcagcagcagcaacagcagcagcagcagcagcagcagcagcaacagcaacagcaacagcaacagcagcaacagcagcaaacccaggccttcagcccacctcctaatgtgactgcttcccccagcatggatgggcttttggcaggacccacaatgccacaagctcctccgcaacagtttccatatcaaccaaattatggaatgggacaacaaccagatccagcctttggtcgagtgtctagtcctcccaatgcaatgatgtcgtcaagaatgggtccctcccagaatcccatgatgcaacacccgcaggctgcatccatctatcagtcctcagaaatgaagggctggccatcaggaaatttggccaggaacagctccttttcccagcagcagtttgcccaccaggggaatcctgcagtgtatagtatggtgcacatgaatggcagcagtggtcacatgggacagatgaacatgaaccccatgcccatgtctggcatgcctatgggtcctgatcagaaatactgctgacatctctgcaccaggacctcttaaggaaaccactgtacaaatgacactgcactaggattattgggaaggaatcattgttccaggcatccatcttggaagaaaggaccagctttgagctccatcaagggtattttaagtgatgtcatttgagcaggactggattttaagccgaagggcaatatctacgtgtttttcccccctccttctgctgtgtatcatggtgttcaaaacagaaatgttttttggcattccacctcctagggatataattctggagacatggagtgttactgatcataaaacttttgtgtcacttttttctgccttgctagccaaaatctcttaaatacacgtaggtgggccagagaacattggaagaatcaagagagattagaatatctggtttctctagttgcagtattggacaaagagcatagtcccagccttcaggtgtagtagttctgtgttgaccctttgtccagtggaattggtgattctgaattgtcctttactaatggtgttgagttgctctgtccctattatttgccctaggctttctcctaatgaaggttttcatttgccattcatgtcctgtaatacttcacctccaggaactgtcatggatgtccaaatggctttgcagaaaggaaatgagatgacagtatttaatcgcagcagtagcaaacttttcacatgctaatgtgcagctgagtgcactttatttaaaaagaatggataaatgcaatattcttgaggtcttgagggaatagtgaaacacattcctggtttttgcctacacttacgtgttagacaagaactatgatttttttttttaaagtactggtgtcaccctttgcctatatggtagagcaataatgctttttaaaaataaacttctgaaaacccaaggccaggtactgcattctgaatcagaatctcgcagtgtttctgtgaatagatttttttgtaaatatgacctttaagatattgtattatgtaaaatatgtatatacctttttttgtaggtcacaacaactcatttttacagagtttgtgaagctaaatatttaacattgttgatttcagtaagctgtgtggtgaggctaccagtggaagagacatcccttgacttttgtggcctgggggaggggtagtgcaccacagcttttccttccccaccccccagccttagatgcctcgctcttttcaatctcttaatctaaatgctttttaaagagattatttgtttagatgtaggcattttaattttttaaaaattcctctaccagaactaagcactttgttaatttggggggaaagaatagatatggggaaataaacttaaaaaaaaatcaggaatttaaaaaaaacgagcaatttgaagagaatcttttggattttaagcagtccgaaataatagcaattcatgggctgtgtgtgtgtgtgtatgtgtgtgtgtgtgtgtgtatgtttaattatgttaccttttcatcccctttaggagcgttttcagattttggttcgtaagacctgaatcccgcggccgccccgggcgtagatactgaaaaaccccgcaagttcacttcaactgtgcatcgtgcaccatctcaatttctttcatttatacatcgttttgccttcttttatgtaactatactcctctaagtttcaatcttggccatgtaacctctgatctatagaattttttaaatgactagaattaatgcccatcttttttttggacctaaattcttcatgaaaatatattacgagggcttattcagaagctttggacttcttcgccagaggtttggtcaagtctccaatcaaggttgtcggcttgtctaccttgccagaaatttacgaaaagatggaaaagggtcaaatcgttggtagatacgttgttgacacttctaaataagcgaatttcttatgatttatgatttttattattaaataagttataaaaaaaataagtgtatacaaattttaaagtgactcttaggttttaaaacgaaaattcttgttcttgagtaactctttcctgtaggtcaggttgctttctcaggtatagcatgaggtcgctcttattgaccacacctctaccggcatgcccgaaattcccctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtaggaaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactacctgtaccccatttaggaccacccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttggggatctcgactctagctagaggatcaattcgtaatcatggtcatagctgtttcctgtgtgaaattgttatccgctcacaattccacacaacatacgagccggaagcataaagtgtaaagcctggggtgcctaatgagtgaggtaactcacattaattgcgttgcgctcactgcccgctttccagtcgggaaacctgtcgtgccagctggattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgtt

SEQ ID NO: 18 - DNA, 7254 bp, artificial sequence
  artificial vector
gcttgcatgcaacttcttttctttttttttcttttctctctcccccgttgttgtctcaccatatccgcaatgacaaaaaaaatgatggaagacactaaaggaaaaaattaacgacaaagacagcaccaacagatgtcgttgttccagagctgatgaggggtatcttcgaacacacgaaactttttccttccttcattcacgcacactactctctaatgagcaacggtatacggccttccttccagttacttgaatttgaaataaaaaaagtttgccgctttgctatcaagtataaatagacctgcaattattaatcttttgtttcctcgtcattgttctcgttccctttcttccttgtttctttttctgcacaatatttcaagctataccaagcatacaatcaactccaagcttgaagcaagcctcctgaaagatgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgccggaattcccgggacagctgcattctcccatcagcaccctgagctcccccatcaacggcatgggcccgcctttctcggtcatcagctcccccatgggcccccactccatgtcggtgcccaccacacccaccctgggcttcagcactggcagcccccagctcagctcacctatgaaccccgtcagcagcagcgaggacatcaagccccccctgggcctcaatggcgtcctcaaggtccccgcccacccctcaggaaacatggcttccttcaccaagcacatctgcgccatctgcggggaccgctcctcaggcaagcactatggagtgtacagctgcgaggggtgcaagggcttcttcaagcggacggtgcgcaaggacctgacctacacctgccgcgacaacaaggactgcctgattgacaagcggcagcggaaccggtgccagtactgccgctaccagaagtgcctggccatgggcatgaagcgggaagccgtgcaggaggagcggcagcgtggcaaggaccggaacgagaatgaggtggagtcgaccagcagcgccaacgaggacatgccggtggagaggatcctggaggctgagctggccgtggagcccaagaccgagacctacgtggaggcaaacatggggctgaaccccagctcgccgaacgaccctgtcaccaacatttgccaagcagccgacaaacagcttttcaccctggtggagtgggccaagcggatcccacacttctcagagctgcccctggacgaccaggtcatcctgctgcgggcaggctggaatgagctgctcatcgcctccttctcccaccgctccatcgccgtgaaggacgggatcctcctggccaccgggctgcacgtccaccggaacagcgcccacagcgcaggggtgggcgccatctttgacagggtgctgacggagcttgtgtccaagatgcgggacatgcagatggacaagacggagctgggctgcctgcgcgccatcgtcctctttaaccctgactccaaggggctctcgaacccggccgaggtggaggcgctgagggagaaggtctatgcgtccttggaggcctactgcaagcacaagtacccagagcagccgggaaggttcgctaagctcttgctccgcctgccggctctgcgctccatcgggctcaaatgcctggaacatctcttcttcttcaagctcatcggggacacacccattgacaccttccttatggagatgctggaggcgccgcaccaaatgacttaggcctgcgggcccatcctttgtgcccacccgttctggccaccctgcctggacgccagctgttcttctcagcctgagccctgtccctgcccttctctgcctggcctgtttggactttggggcacagcctgtcactgctctgcctaagagatgtgttgtcaccctccttatttctgttactacttgtctgtggcccagggcagtggctttcctgagcagcagccttcgtggcaagaactagcgtgagcccagccaggcgcctccccaccgggctctcaggacgccctgccacacccacggggcttgggcgactacagggtcttcggccccagccctggagctgcagccaagctaattccgggcgaatttcttatgatttatgatttttattattaaataagttataaaaaaaataagtgtatacaaattttaaagtgactcttaggttttaaaacgaaaattcttgttcttgagtaactctttcctgtaggtcaggttgctttctcaggtatagcatgaggtcgctcttattgaccacacctctaccggcatgccggcaagtgcacaaacaatacttaaataaatactactcagtaataacctatttcttagcatttttgacgaaatttgctattttgttagagtcttttacaccatttgtctccacacctccgcttacatcaacaccaataacgccatttaatctaagcgcatcaccaacattttctggcgtcagtccaccagctaacataaaatgtaagctttcggggctctcttgccttccaacccagtcagaaatcgagttccaatccaaaagttcacctgtcccacctgcttctgaatcaaacaagggaataaacgaatgaggtttctgtgaagctgcactgagtagtatgttgcagtcttttggaaatacgagtcttttaataactggccttactccaaagacacttcgacgtgactcatcatacaacgtcagaaaacctttatgctcagaaaattattgaccgaaaccgaggaactcttggtattcttgccacgactcatctccatgcagttggacgatatcaatgccgtaatcattgaccagagccaaaacatcctccttaggttgattacgaaacacgccaaccaagtatttcggagtgcctgaactatttttatatgcttttacaagacttgaaattttccttgcaataaccgggtcaattgttctctttctattgggcacacatataatacccagcaagtcagcatcggaatctagagcacattctgcggcctctgtgctctgcaagccgcaaactttcaccaatggaccagaactacctgtgaaattaataacagacatactccaagctgcctttgtgtgcttaatcacgtatactcacgtgctcaatagtcaccaatgccctccctcttggccctctccttttcttttttcgaccgaattaattcgtaatcatggtcatagctgtttcctgtgtgaaattgttatccgctcacaattccacacaacatacgagccggaagcataaagtgtaaagcctggggtgcctaatgagtgaggtaactcacattaattgcgttgcgctcactgcccgctttccagtcgggaaacctgtcgtgccagctggattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagctagaaagtataggaacttcagagcgcttttgaaaaccaaaagcgctctgaagacgcactttcaaaaaaccaaaaacgcaccggactgtaacgagctactaaaaaacttttggttttcgcgagacttctgcgtgaaagttttttggtttttgcgtggcctgacattgctcgatgatttatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgt